(19) United States
(12) Patent Application Publication
(10) Pub. No.: US 2010/0112649 A1
(43) Pub. Date: May 6, 2010

(54) COMPOSITIONS, METHODS AND USES FOR GROWTH OF MICROORGANISMS AND PRODUCTION OF THEIR PRODUCTS

(76) Inventors: Bryan Dennis WILLSON, Fort Collins, CO (US); Gail M. BERARDINO-LANG, LaPorte, CO (US); Amy L. BOCZON, Fort Collins, CO (US); Anna ETTINGER, Thornton, CO (US); Nicholas A. RANCIS, Boulder, CO (US); Mark Thomas MACHACEK, Fort Collins, CO (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/478,374

(22) Filed: Jun. 4, 2009

Related U.S. Application Data
(60) Provisional application No. 61/058,823, filed on Jun. 4, 2008.

(51) Int. Cl. C12P 7/64; C12N 1/00; C12N 1/20; C12N 1/12; C12P 1/00; C12M 1/00

(52) U.S. Cl. 435/134; 435/243; 435/252.1; 435/257.1; 435/41; 435/289.1; 435/257.3; 435/257.4; 435/257.5; 435/257.6

(57) ABSTRACT

Embodiments of the present invention report compositions, methods and uses for growth and/or production of byproducts, products and/or co-products from algal cultures. Certain embodiments of the present invention report using wastewaters to grow microorganisms for producing one or more products, byproducts or co-products from the microorganisms. Other embodiments report compositions and methods for rapid growth of any photosynthetic organism in culture.

### CROSS REFERENCE TO RELATED APPLICATIONS

This application claims the benefit under 35 U.S.C. §119(e) of provisional U.S. patent application Ser. No. 61/058,823 filed on Jun. 4, 2008, incorporated herein by reference in its entirety for all purposes.

### FIELD

Embodiments of the present invention report compositions, methods and uses for growth and/or production of byproducts from algal cultures. Certain embodiments of the present invention report using wastewaters to grow algal cultures for producing one or more products or byproducts. Some embodiments concerning wastewaters report use of brine wastewaters (e.g. produced waters), carbon-rich wastewaters (e.g. inorganic carbon) and/or brewery wastewater for growing microorganisms. Other embodiments report compositions and methods for rapid growth of any photosynthetic organism in culture. Some embodiments report methods for optimizing algal growth to increase biomass and lipid production.

### BACKGROUND

Algae have emerged as one of the most promising sources for biofuels production. Yields of oil from algae can be orders of magnitude higher than traditional oilseeds. One advantage of algae is that algae can grow in places away from the farmlands and forests, minimizing damages caused to the eco- and food-chain systems. In addition, algae, for example, are attractive organisms for biofuel production because they can be grown in and next to treatment plants and/or near power-plant smokestacks where they digest pollutants and produce useful products. With the rise in corn and rice prices, alternative sources of biofuels are becoming even more attractive.

### SUMMARY OF INVENTION

Embodiments of the present invention report compositions, methods and uses for growth and/or production of byproducts from algal cultures. Certain embodiments of the present invention report using wastewaters to grow algal cultures for producing one or more products, byproducts or co-products. Some embodiments concerning wastewaters report use of brine wastewaters (e.g. produced waters), carbon-rich wastewaters and/or brewery wastewater for growing microorganisms.

Other embodiments report compositions and methods for rapid growth of any photosynthetic organisms in culture. In accordance with these embodiments, methods are reported for optimizing algal growth to increase biomass and lipid production at reduced cost and reduced time. Certain embodiments concern high-density, highly scalable methods for algal growth and production. Other embodiments concern detection, identification and increased production of algae byproducts. For example, methods are disclosed for optimizing algal growth to increase biomass and/or lipid production or for example, production of biofuels such as biodiesel, alkanes or other products, byproducts or co-products.

Embodiments herein report methods to increase algae growth to high density levels or to reduce production costs for harvesting or making algal byproducts. For example, these high density growth methods concerns high levels of lipids, including, but not limited to, FAMEs (fatty acid methyl esters) production or TAGs production, where algal cultures can be competitively and cost-effectively grown in AGS (algae growth systems), batches, semi-continuous or continuous culture. In certain embodiments, high-density algal cultures can be generated to increase production of biofuels from algae.

In some embodiments, a composition of the present invention comprises a culture of microorganisms, a brine water composition and at least one supplement. In accordance with these embodiments, the culture of microorganisms can include a culture of photosynthetic organisms or a culture of bacteria. In certain embodiments, a culture of photosynthetic organisms can include one or more cultures of algal strains. Some embodiments can include a brine water composition wherein the brine water composition includes, but is not limited to, produced water.

Other aspects of the present invention may include other compositions for growing and harvesting products from a photosynthetic organism or other microorganisms. In certain embodiments, the composition may include a brewery wastewater composition and one or more cultures of photosynthetic microorganisms. In addition, one or more supplements may be added to the composition. In accordance with these embodiments, the supplements may include a nitrogen supplement, a phosphate supplement, a salt supplement, trace metal supplements or other appropriate supplement for growing the microorganisms. In some embodiments, the culture of photosynthetic organisms may be one or more cultures of algal strains. In other embodiments, a brewery wastewater composition may further include a brine water composition wherein the brine water composition and the brewery wastewater are a mixture of a predetermined ratio or a dilution of water compositions capable of supporting growth of the microorganisms.

Other embodiments may include methods for growing photosynthetic organisms in culture including, but not limited to obtaining a water composition selected from a brine water composition, a brewery wastewater or a mixture thereof and growing the photosynthetic organisms in the water composition. These methods may further include introducing one or more supplements to the culture. In certain embodiments, the photosynthetic organisms may include one or more cultures of algal strains. In addition, these methods may be used for harvesting one or more byproduct, products or co-products from the cultures. Some embodiments may include methods for selecting and culturing photosynthetic organisms that grow more readily in water compositions disclosed herein. For example, a photosynthetic organism that is more tolerant to oil found in produced water or to high concentrations of inorganic carbon. Other embodiments may include methods for selecting and culturing a mutant. These selected cultures may be isolated and prepared for storage for later use or for distribution in a kit or other means for supplying such a culture for future growth and/or expansion. Any photosynthetic organism contemplated in embodiments disclosed herein may be part of a kit for distribution.

Some embodiments concern systems including, but not limited to, providing a brine water composition, a brewery water, other water compositions or combinations thereof, to a culture of microorganisms wherein the system delivers the water to the culture. Some systems may include a sieve or filtering component for removing particulates from the waters (e.g. brine water) prior to the waters contacting the culture. In addition, other systems may include oil separators, ion exchange resins, ozonation, or reverse osmosis for concentration of nutrients. Other systems may include a component for adding one or more supplements to the culture of microorganisms or diluting the water composition (e.g. diluting brine water compositions). In other aspects of the invention, a system may include a culture vessel for culturing the microorganisms and/or a means for harvesting the microorganisms. Yet other embodiments concern systems that deliver waters (e.g. by pumps and pipes) to another area for culturing or storing waters of use in embodiments disclosed herein.

Some embodiments report methods for reducing lag time of photosynthetic organisms may be included. For example, introducing an inoculum of a photosynthetic organism to a media wherein the inoculum and media provide an initial density of about 1 gram wet pellet of algae/liter media to about 5 grams wet pellet of algae/liter media. In accordance with these embodiments, a culture can be in log phase upon inoculation and may be harvested during log or stationary phase. These compositions and methods can result in high biomass yields in a shorter period at reduced costs. Some embodiments, may include methods for harvesting one or more byproducts from these cultures where the time for harvesting a byproduct is reduced. Some embodiments may include but are not limited to, an inoculum from any known species of algae, cyanobacteria or photosynthetic microorganisms. Any embodiment of the present invention may include, but are not limited to, all types of marine or non-marine micro- and picoplankton including, but not limited to, Nannochloropsis oculata, Nannochloropsis sp., Nannochloropsis salina, Nannochloropsis gaditana, Tetraselmis suecica, Tetraselmis chuii, Chlorella sp., Chlorella salina, Chlorella protothecoides, Chlorella ellipsoidea, Chlorella emersonii, Chlorella minutissima, Chlorella pyrenoidosa, Chlorella sorokiniana, Chlorella vulgaris, Chroomonas slaina, Cyclotella cryptic, Cyclotella sp., Dunaliella tertiolecta, Dunaliella salina, Dunaliella bardawil, Botryococcus braunii, Euglena gracilis, Gymnodimium nelsoni, Haematococcus pluvialis, Isochrysis galbana, Monoraphidium minutium, Monoraphidium sp., Nannochloris, Neochloris oleoabundans, Nitzschia laevis, Onoraphidium sp., Pavlova lutheri, Phaeodactylum tricornutum, Porphyridium cruentum, Scenedesmus obliquus, Scenedesmus quadricaula, Scenedesmus sp., Stichococcus bacillaris, Stichococcus minor, Spirulina platensis, Thalassiosira sp., Chlamydomonas reinhardtii, Chlamydomonas sp., Chlamydomonas acidophila, Isochrysis sp., Phaeocystis, Aureococcus, Prochlorococcus, Synechococcus, Synechococcus elongatus, Synechococcus sp., Anacystis nidulans, Anacystis sp., Picochlorum oklahomensis, Picocystis sp. grown separately, or as a combination of species.

Other aspects may include a kit for practicing any of the methods or using any of the compositions disclosed herein. For example, a kit may include one or more algal culture inoculums to achieve an initial culture density of 1 gram of wet pellet/liter media to about 5 grams of wet pellet/liter media (e.g. 1.5 to 2.5 g/L); a media adjusted for growing the culture; one or more containers; and optionally, one or more supplements for supplementing algal media. In some embodiments, nutrient additions to cultures may be about 20% to about 50% less than cultures inoculated with lower concentrations, therefore there can be reduced costs in supplements, time and money. Alternatively, a kit may include one or more algal cultures selected to be more tolerant to water compositions described herein (e.g. grow better in brewery wastewaters or brine waters such as produced water). Kits described of use in certain embodiments may include one or more inoculum of algal cultures disclosed herein. Some kits may include one or more supplement where the supplement is selected from nitrate concentrations of various concentrations and various concentrations of phosphates or salts (e.g. brine waters or instant ocean or other salt supplements) depending on for example, water compositions used, microorganisms grown, density of culture and light exposure etc. Kits disclosed herein may be used to culture microorganisms of use as products, for harvesting byproducts or co-products or combinations thereof.

In certain embodiments, compositions for increasing lipid production in an algal culture are disclosed. For example, media for growing algal cultures can have a calcium precipitant to precipitate calcium out of the media and/or have no supplemental calcium compound in the media.

Some aspects report methods for detecting lipid production by algal cultures are reported herein. For example, one or more fluorescent dyes may be used to detect lipids produced by algal cultures. Certain embodiments may include the use of fluorescent dyes such as Nile Red or Bodipy stain. Kits are also contemplated of use for a more portable mean of detecting lipid production by an algal culture. A kit may include, but is not limited to, a container means for containing a sample and at least one fluorescent dye.

Other embodiments herein can include methods for obtaining lipids by, for example, lipid extraction, transesterification, base catalyzed transesterification, and/or methyl esterifrication to obtain any type of lipids (e.g. FAMEs). Any method known in the art for harvesting and/or isolating lipids is contemplated of use herein.

Certain embodiments concern compositions for growing algae. Some embodiments concern compositions of algal media for growing, for example any marine algae, including, but not limited to, Nannochloropsis oculata, Nannochloropsis gaditana, Nannochloropsis salina, Nannochloropsis sp. Other embodiments concern identifying and or selecting algal cultures for increasing production of algal lipids.

Some embodiments concern compositions and methods for growing algae at reduced costs by reducing the frequency of feeding algal cultures and changing the composition of algal feed. For example, Nannochloropsis oculata can be grown on a single dose of a composition including, but may not be limited to, nitrates, phosphates and trace metals. In some embodiments, growth using a single dose can be from about 4 to about 30 days, for example, by batch culture. In certain embodiments, a culture can be continuously fed, or not continuously fed. Some embodiments concern using a single dose of a nitrogen supplement (e.g. KNO₃ or NaNO₃ or NH₄NO₃) that can be about 0.05-1.5 gms/L and/or a single dose of phosphates (KH₂PO₄ or K₂HPO₄ or Na₂HPO₄ or NaH₂PO₄) that can be about 0.02-0.1 gms/L to grow algal cultures, for example, Nannochloropsis oculata. Nutrient addition to cultures can depend on light exposure and growth in an indoor facility or an outdoor facility. Some algal cultures grown by methods disclosed herein can include a density of about 1.0-10 g/L with about 20-50% of FAMEs. In some embodiments, growth using a single dose can be from about 4 to about 30 days to produce lipids such as FAMEs. Thus, in accordance with these embodiments, a minimal to no-feed approach may be used for growing algae, reducing or eliminating a need for additional supplementation of nutrients to the cultures, saving money, labor and time. In addition, these embodiments reduce or eliminate a need for some depletion stages. In accordance with these embodiments, algae can use most or all nutrients from media within 1-7 days (e.g. nitrogen compound, phosphate compound), by use or storage for later use, and generate co-products and/or lipids close to or at the same time as nutrient depletion. These methods of a reduced feed approach can induce or increase production of algal products, byproducts or co-products for example, lipids and amino acids. In certain embodiments, some algae may contain relatively high amounts of lipids compared to a control that can be converted to biofuels during log-phase growth.

Other embodiments, may concern nutrient depletion of photosynthetic microorganism cultures to increase lipid production from the algal cultures. Some embodiments concern introduction of nutrients to a culture proportional to algae biomass until a predetermined biomass amount is attained, for example, a maximum level of biomass is obtained from the algal culture. Any of these embodiments may be used in combination with other compositions, methods and systems disclosed herein.

### BRIEF DESCRIPTION OF THE DRAWINGS

The following drawings form part of the instant specification and are included to further demonstrate certain aspects of particular embodiments herein. Embodiments may be better understood by reference to one or more of these drawings in combination with the detailed description presented.

FIG. 1 is a bar graph representing biomass production in algal cultures grown in various medias.

FIG. 2 are comparative graphs representing biomass production in algal cultures grown in various medias over several days.

FIG. 3 are comparative graphs representing % FAMEs production in algal cultures grown in various medias at 2 different days of harvest.

FIG. 4 is a graph representing biomass production in algal cultures grown in various medias over several days.

FIG. 5 is a graph representing biomass production in algal cultures grown in various medias with different salt supplements over several days.

FIG. 6 is a graph representing biomass production in different batches of algal cultures grown out of doors in a brine water over several days.

FIG. 7 is a graph representing % FAMEs harvested over several days in various batches of algal cultures grown in produced water.

FIG. 8 is a table representing % FAMEs produced in various batches of algal organisms grown in produced water.

FIG. 9 is a graph representing pH changes in media overtime of algal growth exposed to different nitrogen sources in different media compositions.

FIG. 10 is a graph representing algal culture growth over several days in a various media.

FIG. 11 is a bar graph representing % FAMEs produced by algal cultures grown over several days in a various media.

FIG. 12 is a bar graph representing lipid production in algal cultures grown in various media compositions.

### DESCRIPTION OF ILLUSTRATIVE EMBODIMENTS

### Definitions

As used herein, “a” or “an” may mean one or more than one of an item.

As used herein, “about” may mean up to and including plus or minus five percent, for example, about 100 may mean 95 and up to 105.

As used herein “introduced” (not native, obtained elsewhere) microorganism can mean a microorganism introduced by human manipulation, mechanical manipulation or systematic manipulation. For example, an introduced bacterial culture or an introduced algae culture can be a microorganism introduced to a disclosed water composition for producing products of the microorganism for its products, byproducts or co-products (e.g. Nannochloropsis sp.).

As used herein “brine” may mean water saturated or nearly saturated with common salt or a strong saline solution or water of a sea or salt lake. As used herein “produced water” may mean brine waters of the United States or another country. For example, United States produced water can be defined by the Environmental Protection Agency as, for example, water (brine) brought up from the hydrocarbon bearing formation strata during the extraction of oil and gas, and can include formation water, injection water, and any chemicals added downhole or during the oil/water separation process.

As used herein “produced water” may mean brines that flow or are lifted to the surface with oil, or water that is generated along with oil and gas. In some produced waters, sodium chloride may be a prominent salt and soil, grease, oil, ethyl benzene, benzene, phenols and toluene may be some of the organic contaminants.

As used herein “brewery wastewater” may mean any type of brewery wastewater, microbrewery wastewater, large-scale brewery wastewater, brewery discard water, pre- or post-water treatment, but that has not been exposed to cleaning, disinfecting or sanitizing agents such as bases, acids, detergents or iodophores.

As used herein “modulate” can mean a change in levels or concentrations of an agent, byproduct or molecule, a change can be up or down. For example, a modulation of byproduct production in a photosynthetic organism can mean the levels go up.

### DETAILED DESCRIPTION

In the following sections, various exemplary compositions and methods are described in order to detail various embodiments. It will be obvious to one skilled in the art that practicing the various embodiments does not require the employment of all or even some of the specific details outlined herein, but rather that concentrations, times and other specific details may be modified through routine experimentation. In some cases, well-known methods or components have not been included in the description.

Embodiments of the present invention report compositions, methods, systems and apparatus for culturing and using photosynthetic organisms. Other embodiments report, compositions for growing and using photosynthetic microorganisms in remote areas. Certain embodiments concern growing algae in waste products, for example liquid wastes such as wastewaters produced by various industries (e.g. brine wastewater and brewery waters). In addition, other embodiments concern use of algal products such as lipids and other byproducts for producing biofuels. In accordance with these embodiments, algal cultures may be induced to produce increased levels of byproducts (e.g. lipids) or use wastewaters for reduced cost methods for producing the byproducts. Byproducts, for example, include, but are not limited to feed for marine and land animals, lipids, amino acids, carbohydrates, alkanes and byproducts for biofuel products may be generated from algal cultures. For example, other products may include protein or amino acid compositions generated from algal cultures disclosed herein.

Embodiments of the present invention report compositions, methods and uses for growth and/or production of byproducts from algal cultures. Certain embodiments of the present invention report using wastewaters to grow algal cultures for producing one or more products. Some embodiments concerning wastewaters report use of brine wastewaters (e.g. produced waters), carbon-rich wastewaters (e.g. inorganic carbon such as bicarbonate) and/or brewery wastewater for growing microorganisms.

Other embodiments report compositions and methods for rapid growth of any photosynthetic organisms in culture. In accordance with these embodiments, methods are reported for optimizing algal growth to increase biomass and lipid production at reduced cost and reduced time. Certain embodiments concern high-density, highly scalable methods for algal growth and production. Other embodiments concern detection, identification and increase production of algae byproducts. For example, methods are disclosed for optimizing algal growth to increase biomass and/or lipid production or for example, production of biofuels such as biodiesel at a reduced cost in time and money.

Embodiments can include methods to increase algae growth to high density levels or to reduce production costs for harvesting or making algal byproducts. For example, these high density growth methods may concern similar or modulated levels of FAMEs (fatty acid methyl esters) production or TAGs, where algal cultures can be competitively and cost-effectively grown in AGS (algae growth systems), batches, semi-continuous or continuous culture. In certain embodiments, high-density algal cultures can be generated to increase production of biofuels from algae. In accordance with these embodiments, algal cultures may be grown by an initial high density inoculum to reduce growth and production times.

In some embodiments, a composition of the present invention comprises a culture of microorganisms, a brine water composition and at least one supplement. In accordance with these embodiments, the culture of microorganisms can include a culture of photosynthetic organisms or a culture of bacteria. In certain embodiments, a culture of photosynthetic organisms can include one or more cultures of algal strains. Algal strains that may be grown in these compositions, include, but are not limited to, Nannochloropsis oculata, Nannochloropsis sp., Nannochloropsis salina, Nannochloropsis gaditana, Tetraselmis suecica, Tetraselmis chuii, Chlorella sp., Chlorella salina, Chlorella protothecoides, Chlorella ellipsoidea, Chlorella emersonii, Chlorella minutissima, Chlorella pyrenoidosa, Chlorella sorokiniana, Chlorella vulgaris, Chroomonas slaina, Cyclotella cryptic, Cyclotella sp., Dunaliella tertiolecta, Dunaliella salina, Dunaliella bardawil, Botryococcus braunii, Euglena gracilis, Gymnodimium nelsoni, Haematococcus pluvialis, Isochrysis galbana, Monoraphidium minutium, Monoraphidium sp., Nannochloris, Neochloris oleoabundans, Nitzschia laevis, Onoraphidium sp., Pavlova lutheri, Phaeodactylum tricornutum, Porphyridium cruentum, Scenedesmus obliquus, Scenedesmus quadricaula, Scenedesmus sp., Stichococcus bacillaris, Stichococcus minor, Spirulina platensis, Thalassiosira sp., Chlamydomonas reinhardtii, Chlamydomonas sp., Chlamydomonas acidophila, Isochrysis sp., Phaeocystis, Aureococcus, Prochlorococcus, Synechococcus, Synechococcus elongatus, Synechococcus sp., Anacystis nidulans, Anacystis sp., Picochlorum oklahomensis, Picocystis sp. grown separately, or as a combination of species.

Bacterial strains that may be grown in compositions disclosed, include, but are not limited to, Pseudomonas sp., E. coli or other species tolerant of diesel range organics and growth in organic carbons sources provided by growing algae. Some embodiments can include a brine water composition wherein the brine water composition includes, but is not limited to, produced water. It is contemplated herein that water compositions disclosed may be used to grow microorganisms in any location including, but not limited to, where these waters are found or in an area removed from these where the waters are collected.

Other aspects of the present invention may include other compositions for growing and harvesting products from a photosynthetic organism or other microorganisms. In certain embodiments, a composition may include a brewery wastewater composition and one or more cultures of photosynthetic microorganisms. In addition, one or more supplements may be added to the composition. In accordance with these embodiments, the supplements may include a nitrogen supplement, a phosphate supplement, a salt supplement or other appropriate supplement for growing the microorganisms. In some embodiments, the culture of photosynthetic organisms may be one or more cultures of algal strains. In other embodiments, a brewery wastewater composition may further include a brine water composition wherein the brine water composition and the brewery wastewater are a mixture of a predetermined ratio capable of growing the culture microorganisms. In accordance with these embodiments, a ratio of brine water composition to brewery water composition may be any ratio necessary to achieve a predetermined media/nutrient content, for example a ratio of 1 to 1, 1 to 2, 1 to 3, 1 to 4, 1 to 5, etc or fractions thereof or mixtures thereof. Conversely, a ratio of brine water composition to brewery water composition may be 2 to 1, 3 to 1, 4 to 1, 5 to 1, 6 to 1 etc. or fractions thereof or mixtures thereof. These compositions can further comprise one or more supplements in order to promote optimum growth of the organisms. Supplements contemplated of use in these compositions may include, but are not limited to, nitrogen, salts, phosphates, or other media supplement.

Other embodiments may include methods for growing photosynthetic organisms in culture including, but not limited to obtaining a water composition selected from a brine water composition, a brewery wastewater or a mixture thereof and growing the photosynthetic organisms in the water composition. These methods may further include introducing one or more supplements to the culture. In certain embodiments, the photosynthetic organisms may include one or more cultures of algal strains. In addition, these methods may be used for harvesting one or more byproduct from the cultures. Some embodiments may include methods for selecting and culturing photosynthetic organisms that grows more readily in water compositions disclosed herein. For example, a photosynthetic organism that is more tolerant to oil or bicarbonate or other high level of inorganic carbon found in produced water. Other embodiments may include methods for selecting and culturing a mutant. These selected cultures may be isolated and prepared for storage for later use or for distribution in a kit or other means for supplying such a culture for future growth and/or expansion. Any photosynthetic organism contemplated in embodiments disclosed herein may be part of a kit for distribution.

In one aspect of the present invention, mutagenesis may be used to identify, isolate and culture an organism adapted to grow more readily in compositions of the present invention and/or produce higher levels of byproducts in compositions of the present invention and/or grow more rapidly in compositions of the present invention. In accordance with these embodiments, procedures for identifying and obtaining microorganisms or mutant microorganisms capable of adapting to these compositions are known in the art. For example, classical genetics techniques can be used to obtain improved strains of a microalgal species. Any method known in the art can be used including, but not limited to, random mutagenesis, isolation of spontaneous mutants or other method for obtaining mutant cultures. Certain methods may include, but are not limited to random mutagenesis techniques such as any type of irradiation or application of a chemical agent with subsequent screening or selection methods known in the art. In accordance with these embodiments, improvements include, but are not limited to, modulated growth, reduced lag time, modulated fatty acid profile, lipid content, carbohydrate levels, sugar content, protein content, pigment content, co-factor content, cell wall composition, cell wall strength or any combination thereof.

Some embodiments concern systems including, but not limited to, providing a brine water composition, a brewery water or other water composition to a culture of microorganisms wherein the system delivers the water to the culture. In accordance with these embodiment, a system may include tubes, pipes, or other means for delivering compositions of the present invention to microorganism cultures. Some systems may include a sieve or filtering component for removing particulates from the waters (e.g. brine water, brewery wastewater), a component for removing unwanted ions, reverse osmosis for concentrating nutrients or a component for rapid screening of unwanted chemicals prior to the waters contacting the culture. It is contemplated that a sieve system may remove debris that affects the growth of the organism while preserving components, nutrients, minerals or agents that modulate the growth of the organisms. Other systems may include a component for adding one or more supplements to the culture of microorganisms. In other aspects of the invention, a system may include a culture vessel for culturing the microorganisms and/or a means for harvesting the microorganisms. Yet other embodiments concern systems that deliver waters (e.g. by pumps and pipes) to another area or region away from a site where the wastewater or brine water is obtained. For example, an area may be at the site or at a neighboring site near a natural gas system, an oil field, an off-shore oil rig or a brewery. Alternatively, the brine water or wastewater compositions may be removed by transferring the water compositions to a separate area than where the waters are obtained, for use or storage for later use.

In other embodiments, methods for reducing lag time for growing photosynthetic organisms may be included. For example, introducing an inoculum of a photosynthetic organism to a media wherein the initial culture density is about 1 gram/liter to about 5 grams/liter (e.g. 1.5 to 2.5 gms/L) which may put the organism nearing or directly into log phase upon inoculation; and permit harvesting the algal culture in log or stationary phase in a reduced period of time. The methods may include harvesting one or more byproducts from these cultures where the time for harvesting a byproduct is reduced. Some embodiments can include, but are not limited to an inoculum of Nannochloropsis oculata, Nannochloropsis sp., Nannochloropsis salina, Nannochloropsis gaditana, Tetraselmis suecica, Tetraselmis chuii, Chlorella sp., Chlorella salina, Chlorella protothecoides, Chlorella ellipsoidea, Chlorella emersonii, Chlorella minutissima, Chlorella pyrenoidosa, Chlorella sorokiniana, Chlorella vulgaris, Chroomonas slaina, Cyclotella cryptic, Cyclotella sp., Dunaliella tertiolecta, Dunaliella salina, Dunaliella bardawil, Botryococcus braunii, Euglena gracilis, Gymnodimium nelsoni, Haematococcus pluvialis, Isochrysis galbana, Monoraphidium minutium, Monoraphidium sp., Nannochloris, Neochloris oleoabundans, Nitzschia laevis, Onoraphidium sp., Pavlova lutheri, Phaeodactylum tricornutum, Porphyridium cruentum, Scenedesmus obliquus, Scenedesmus quadricaula, Scenedesmus sp., Stichococcus bacillaris, Stichococcus minor, Spirulina platensis, Thalassiosira sp., Chlamydomonas reinhardtii, Chlamydomonas sp., Chlamydomonas acidophila, Isochrysis sp., Phaeocystis, Aureococcus, Prochlorococcus, Synechococcus, Synechococcus elongatus, Synechococcus sp., Anacystis nidulans, Anacystis sp., Picochlorum oklahomensis, Picocystis sp. grown separately, or as a combination of species. In certain embodiments Nannochloropsis oculata, Nannochloropsis sp., Nannochloropsis salina, may be grown using a water composition having modulated inorganic carbon content.

Other aspects of the present invention can include kits for practicing any of the methods or using any of the compositions disclosed herein. For example a kit may include one or more algal culture inoculums sufficient to achieve an initial inoculation of about 1 gram/liter to about 5 grams/liter; one or more containers; and optionally, one or more supplements for supplementing algal media. Alternatively, a kit may include one or more algal cultures selected to be more tolerant for growth in waters of compositions described herein (e.g. brewery wastewaters or brine waters such as produced water). In addition, kits may include one or more modified medias, lyophilized for ready rehydration. Kits described of use in certain embodiments may include one or more inoculums of algal cultures selected from Nannochloropsis oculata, Nannochloropsis sp., Nannochloropsis salina, Nannochloropsis gaditana, Tetraselmis suecica, Tetraselmis chuii, Chlorella sp., Chlorella salina, Chlorella protothecoides, Chlorella ellipsoidea, Chlorella emersonii, Chlorella minutissima, Chlorella pyrenoidosa, Chlorella sorokiniana, Chlorella vulgaris, Chroomonas slaina, Cyclotella cryptic, Cyclotella sp., Dunaliella tertiolecta, Dunaliella salina, Dunaliella bardawil, Botryococcus braunii, Euglena gracilis, Gymnodimium nelsoni, Haematococcus pluvialis, Isochrysis galbana, Monoraphidium minutium, Monoraphidium sp., Nannochloris, Neochloris oleoabundans, Nitzschia laevis, Onoraphidium sp., Pavlova lutheri, Phaeodactylum tricornutum, Porphyridium cruentum, Scenedesmus obliquus, Scenedesmus quadricaula, Scenedesmus sp., Stichococcus bacillaris, Stichococcus minor, Spirulina platensis, Thalassiosira sp., Chlamydomonas reinhardtii, Chlamydomonas sp., Chlamydomonas acidophila, Isochrysis sp., Phaeocystis, Aureococcus, Prochlorococcus, Synechococcus, Synechococcus elongatus, Synechococcus sp., Anacystis nidulans, Anacystis sp., Picochlorum oklahomensis, Picocystis sp. supplied separately, or as a combination of species. Some kits may include one or more supplement where the supplement is selected from nitrate concentrations of 0.1 to 1.5 grams per liter, various concentrations of phosphates, trace metals, nitrogen sources or salts (e.g. brine waters or other salt supplements). Other supplements may include micro and/or macro nutrients from any source including, but not limited to, commercial fertilizers, commercial salts or wastewaters.

Some methods of use for detecting lipid production by algal cultures are reported herein. For example, one or more fluorescent dyes may be used to detect lipids produced by photosynthetic organisms. Certain embodiments may include the use of fluorescent dyes such as Nile Red or Bodipy stain to analyze lipid content of algal cultures. Kits are also contemplated of use for a more portable mean for detecting lipid production by an algal culture. A kit may include, but is not limited to, a container means for containing a sample and at least one fluorescent dye. In addition, a means for detecting fluorescence produced by these dyes is contemplated. In certain kits, a detection instrument having portability is contemplated for a quick analysis of byproduct production by for example, algal microorganisms.

Any apparatus or method known in the art may be used to grow microorganisms of the embodiments of the present invention. Certain methods herein concern using closed system photobioreactors to grow photosynthetic organisms (e.g. algae). Other methods herein concern using open system photobioreactors to grow photosynthetic organisms (e.g. algae). Methods of use for culturing photosynthetic organisms can include batch, semi-batch, semi-continuous, plug batch, incremental feed, biphasic, gas sparged (e.g. flue gas, carbon dioxide and/or air) or no gas introduction to cultures, continuous, partial or not mixed culturing technologies or any other method for culturing known in the art. In accordance with these embodiments, these systems can be located on essentially underutilized parcels of land without growth (e.g. on top of a building), water or nutrient competition with other food crops. For example, an AGS system can be set up in a sea using sea waves as source of continuous shaking, near an ocean where produced water is utilized from an offshore oilrig, or in arid regions where natural gas or other oils are sought after. Photosynthetic organisms can grow all year long and quickly replace extracted byproducts, products or co-products, therefore year-round harvests are contemplated in any area or location. In other embodiments, algae or microalgae can utilize carbon dioxide-rich flue gases of fossil-fuel power plants and other industrial exhaust gases as sources of carbon dioxide. In accordance with these embodiments, utilizing water compositions disclosed for growing photosynthetic organisms can decrease the quantity of greenhouse gases expelled into the atmosphere from power plants, sewer plants, breweries, and wineries, as well as serve as a way for producing useful byproducts of the organisms.

Containment methods contemplated of use herein may include, but is not limited to, bags, panels, ponds, pools, flasks, tubular bioreactors, fermentors or other culturing vessel known in the art. Methods for harvesting microorganisms or byproducts of microorganisms can include centrifugation, spray drying, filtration, flocculation, ultrasound or any other harvesting methods known in the art. Byproducts, co-products or products that may be derived from a photosynthetic culture may include, but are not limited to, whole live cultures for propagation, distribution or feed, whole organisms or fragments of the organism or components of the organisms, for example, lipids, nutraceuticals, cosmetics, toiletries, carbohydrates, amino acids, proteins or others known in the art. In certain embodiments, these products may be used to generate other products or used for human or animal consumption or products.

In some embodiments, one or more photosynthetic species may be cultured, within a single culturing system such as a photobioreactor in any of the compositions disclosed. In other embodiments, one or more microorganisms may be cultured together, for example, photosynthetic microbes and bacterial microbes. In certain embodiments, one or more of the photosynthetic species may be the predominant organism in a culture when weather and environmental conditions favor its growth over growth the photosynthetic species. In accordance with these embodiments, algal cultures can include, but are not limited to, Nannochloropsis oculata, Nannochloropsis sp., Nannochloropsis salina, Nannochloropsis gaditana, Tetraselmis suecica, Tetraselmis chuii, Chlorella sp., Chlorella salina, Chlorella protothecoides, Chlorella ellipsoidea, Chlorella emersonii, Chlorella minutissima, Chlorella pyrenoidosa, Chlorella sorokiniana, Chlorella vulgaris, Chroomonas slaina, Cyclotella cryptic, Cyclotella sp., Dunaliella tertiolecta, Dunaliella salina, Dunaliella bardawil, Botryococcus braunii, Euglena gracilis, Gymnodimium nelsoni, Haematococcus pluvialis, Isochrysis galbana, Monoraphidium minutium, Monoraphidium sp., Nannochloris, Neochloris oleoabundans, Nitzschia laevis, Onoraphidium sp., Pavlova lutheri, Phaeodactylum tricornutum, Porphyridium cruentum, Scenedesmus obliquus, Scenedesmus quadricaula, Scenedesmus sp., Stichococcus bacillaris, Stichococcus minor, Spirulina platensis, Thalassiosira sp., Chlamydomonas reinhardtii, Chlamydomonas sp., Chlamydomonas acidophila, Isochrysis sp., Phaeocystis, Aureococcus, Prochlorococcus, Synechococcus, Synechococcus elongatus, Synechococcus sp., Anacystis nidulans, Anacystis sp., Picochlorum oklahomensis, Picocystis sp. grown separately, or as a combination of species. In certain embodiment, such as during summer months when solar illumination and environmental temperature are at a maximum, certain species may be a predominant culture. In other embodiments, during milder fall and spring months, other microorganisms may dominate a culture. In addition, during colder winter months, Nannochloropsis ability to thrive at very low temperatures may allow it to dominate a culture. Each species would thus be present at all times of the year, but the proportions of different species will vary seasonally. It may also be beneficial because some algae species can release antibacterial substances thereby naturally fighting bacterial contamination. In other aspects of the invention more than one organism may be cultured in water compositions contemplated herein, for example bacteria, photosynthetic organisms, protozoa and fungi may be cultured separately or in any combination. In one example, pseudomonas may be cultured at the same time or different times in the same water composition as an algal culture (e.g. brewery wastewater, brine water). Some aspects of the present invention concern photosynthetic and bacterial species disclosed herein, alone or in combination with another species, can be an introduced microorganism to water compositions described.

### Brewery Wastewater

In certain exemplary methods, wastewater may be used as a media for the growth of algal cultures. In some embodiments, a wastewater may be a brewery wastewater. It has been found that wastewater of a brewer is a significant waste products of brewery operations often in the range of 3-10 liters of waste for every liter of beer generated. In certain embodiments, for example, microbrewery wastewater, large-scale brewery wastewater or a combination may be used to grow algal cultures. Photosynthetic organisms capable of growing in wastewater include, but are not limited to, Nannochloropsis oculata, Nannochloropsis sp., Nannochloropsis salina, Nannochloropsis gaditana, Tetraselmis suecica, Tetraselmis chuii, Chlorella sp., Chlorella salina, Chlorella protothecoides, Chlorella ellipsoidea, Chlorella emersonii, Chlorella minutissima, Chlorella pyrenoidosa, Chlorella sorokiniana, Chlorella vulgaris, Chroomonas slaina, Cyclotella cryptic, Cyclotella sp., Dunaliella tertiolecta, Dunaliella salina, Dunaliella bardawil, Botryococcus braunii, Euglena gracilis, Gymnodimium nelsoni, Haematococcus pluvialis, Isochrysis galbana, Monoraphidium minutium, Monoraphidium sp., Nannochloris, Neochloris oleoabundans, Nitzschia laevis, Onoraphidium sp., Pavlova lutheri, Phaeodactylum tricornutum, Porphyridium cruentum, Scenedesmus obliquus, Scenedesmus quadricaula, Scenedesmus sp., Stichococcus bacillaris, Stichococcus minor, Spirulina platensis, Thalassiosira sp., Chlamydomonas reinhardtii, Chlamydomonas sp., Chlamydomonas acidophila, Isochrysis sp., Phaeocystis, Aureococcus, Prochlorococcus, Synechococcus, Synechococcus elongatus, Synechococcus sp., Anacystis nidulans, Anacystis sp., Picochlorum oklahomensis, Picocystis sp. grown separately, or as a combination of species.

Brewery wastewater can contain elevated levels of various types of nutrients such as nitrogen, phosphorous and trace metals and other micronutrients. Therefore, in some embodiments, with minor adjustments, it can be used to grow microorganisms such as growing photosynthetic microorganisms for products such as biofuels. Brewery wastewater can also be used in combination with brine waters. Certain embodiments disclose methods and compositions for growing microorganisms in brewery wastewater using any method of nutrient addition, inoculation and growth that is known in the art including, but not limited to batch, semi-batch, continuous, semi-continuous, plug batch, incremental feed, fed-batch, biphasic production sparged with air, flue gas, carbon dioxide and using various types of mixing. Supplemental nutrient addition to the brewery wastewater with macro- and/or micronutrients can come from any source, including commercial fertilizers, commercial salts or other wastewater streams. Production can be performed in any type of container common in the art including, but not limited to, bags, panels, ponds, pools, flasks, tubular bioreactors, fermentors and any other container. In addition, cultures may be harvested by any method known in the art including, but not limited to, centrifugation, spray drying, filtration, drum drying, settling, flocculation, ultrasound, acoustic focusing, hydrodynamic focusing and other harvesting methods known. Uses for algae, algae products, byproducts and co-products can include any use common in the art including, but not limited to, whole live algae for propagation, inoculation or feed, whole dead algae, or any part thereof, such as lipids for biofuels, nutraceuticals, cosmetics, toiletries, carbohydrates for fermentation or pulp, or proteins for human or animal feed.

In some embodiments, microbrewery wastewater (e.g NBB) may be used. In one example, a microbrewery's waste stream is about 83,000 gallons per day containing an average of 25 ppm phosphorus as phosphate and an average of 50 ppm (mg/L) nitrogen as nitrate. The nitrate concentrations ranged depending on what type of beer was brewed and was highest in certain months of the year, including, March-April and June-July. Total suspended solids of the water had an average of 21.0 mg/L. The conductivity of the waste water averaged about 898 mg/L sodium and the conductivity was the highest from March through August. In some embodiments, brewery wastewater may be used in certain months of the year in order to take advantage of elevated supplement levels. In other embodiments, brewery wastewater may be used during any month of the year for growing photosynthetic organisms. In addition, brewery wastewater may be used to supplement cultures of photosynthetic microorganisms grown in other waters.

### Produced Water

In other embodiments, microorganisms contemplated herein may be grown in a wastewater produced from extracting earth fuels. In certain embodiments, a brine water composition can be a produced water composition of use for growing microorganisms of the present invention. With the ratio of water to oil getting more and more unbalanced, produced water treatment is becoming more important. One problem is that produced water is high in inorganic carbon and other contaminants, so reusing this water requires extensive and expensive treatment processes. Produced water comes from the process of lifting oil and gas from water-bearing formations—typically ancient sea or lakebeds. As oil and gas is lifted to the surface, water is brought along with it.

Some embodiments of the present invention concern using produced waters of the U.S and other countries. Produced waters of the U.S. have been analyzed and about 65% is injected back into the producing formation, 30% into deep saline formations and 5% is discharged to surface waters. Produced water salinity in the U.S. ranges from 100 mg/l to 400,000 mg/l (seawater is 35,000 mg/l). Produced water is mainly salty water trapped in the reservoir rock and brought up along with oil or gas during production. It can contain very minor amounts of chemicals added downhole during production. These waters exist under high pressures and temperatures, and usually contain oil and metals. Because of these contaminants, these waters are typically treated prior to being discharged. Treatment of produced water is a major component of the cost of producing oil and gas. Wells may start out having a low level of produced water but eventually all oil wells produce excess volumes of water to oil. Efficient and economical disposal of produced water is critical to the success in the oil production business. Produced water is either reused or disposed of. Embodiments herein illustrate uses of produced water compositions for growing or supplementation of growth of photosynthetic or bacterial organisms (e. g. algal cultures or bacterial cultures (e.g. E. coli, Pseudomonas)).

In certain embodiments, produced waters contain some subset or mixture of the following components or agents depending on the oil or gas produced and the region of production and added agent for production of the oil or gas. For example, produced gas can contain dissolved inorganic salts, dispersed oil droplets, dissolved organic compounds (dissolved “oil”), treatment and workover chemicals, dissolved gases (e.g. hydrogen sulfide and carbon dioxide), and other contaminants for example, soil, oil, grease, ethyl benzene, benzene, phenols and toluene. Chemical equilibrium systems can shift in produced water with changes in temperature and pressure and cause reactions to occur. These reactions may result in mineral scales being formed, solid hydrocarbon deposition (paraffin formation) and changes in pH. Deposition of iron compounds and elemental sulfur may also be found in produced waters. It is contemplated herein that any produced water may be used for growing any photosynthetic organism or bacterial organism disclosed. For example, produced water may be used as a basis for a media for these microorganisms or as supplemental compositions to another media composition, such as a brewery water composition. Produced water may be used as generated, diluted, or it may be treated or pre-treated by removing some or all of the contaminants that can affect the growth of or byproduct formation from the microorganisms

Some embodiments concern growth of photosynthetic microorganisms using produced water compositions disclosed and methods for growing these microorganisms at reduced costs. In accordance with these embodiments, methods and compositions may include reduced frequency of feeding cultures and/or changing the composition of media or feed such as batch or continuous culture. For example, certain embodiments concern an interval or single-feed approach to culturing algae. In one exemplary method, algae (e.g. Nannochloropsis oculata) can be grown on a single dose of nitrates, phosphates and trace metals for about 6 to about 10 days including growth and harvesting. In accordance with some embodiments, a culture can be continuously fed, or incrementally fed. In certain embodiments, a single dose of nitrogen (KNO₃ or NaNO₃ or NH₄NO₃) can be about 0.1-1.5 g/L and a single dose of phosphates (KH₂PO₄ or K₂HPO₄ or Na₂HPO₄ or NaH₂PO₄) can be about 0.02-0.05 g/L to grow photosynthetic organisms. For example, some algal cultures grown by methods disclosed herein can include a density of about 3.5-10 g/L with about 20-50% of FAMEs in about 4-30 days. Thus, these embodiments concerning a minimal to no-feed approach can be a cost-effective way to grow photosynthetic organism by reducing or eliminating a need for additional supplementation of the cultures, saving money, labor and time for batch approach. In addition, these embodiments reduce or eliminate a need for additional depletion stages. In certain embodiments, these methods of reduced feed may induce production of algal products, co-products or byproducts, for example, lipids. Lipids form when algae are growing with a peak formation when nutrients are significantly reduced or depleted.

Other embodiments concern compositions, methods and uses for inducing increased levels of lipid production in a short time period using incremental feeding of algae. In accordance with these methods, algae may be fed on a daily incremental basis to get lipid formation in a very short time. This time period may be 2-5 days, or 3-4 days, or 2-3 days, from the beginning of algal cultivation. In other embodiments, nutrients may be depleted from an algal culture after a feeding and this feeding regimen may be repeated one or more times, depleting nutrients to increase lipid production from the algal cultures. Some embodiments concern introduction of nutrients to a culture proportional to algae biomass until a predetermined biomass amount is attained, for example, a maximum level of biomass is obtained from the algal culture.

### Lipid Detection In Algae

Other embodiments herein concern improved lipid detection techniques. In accordance with these embodiments, lipid detection techniques herein can have reduced false positives and/or improved detection of lipids produced by algae cultures. In one exemplary embodiment, an improved lipid detection technique can include a fluorescent dye, for example, Nile Red or Bodipy. In order to reduce false positives, algal cells may be washed with media, pelleted and resuspended in fresh media. In certain embodiments, algae cultures may be heat shocked and/or exposed to osmotic stress for increasing or facilitating lipid detection by fluorescent dyes. In other embodiments, preparation for measuring lipids in an algal culture can be performed in glass containers to avoid adherence to plastics. In accordance with these embodiments, a signal may be measured at excitation 525 nm and emission 580 nm in order to assess lipid production of the algae. In certain embodiments, another analysis may include detection of neutral lipids at 540-600 nm, and polar lipids and chlorophyll that emit at a wavelength above 600 nm. Algae can pack neutral lipids into cytoplasmic vesicles or lipid bodies. Neutral lipids represent esterified fatty acid fraction of cells, triacylglycerols. Polar lipids in the form of phospholipids and glyco lipids dominate cell membranes and fluoresce in orange spectrum. Polar lipids contain a shorter chained, saturated fatty acid.

In other aspects of the present invention, a method for detecting lipid production by algal microorganisms may be used, including, but not limited to using one or more fluorescent dyes to detect lipids produced by the algal microorganisms. In accordance with these embodiments, a fluorescent dye can include any fluorescent dye capable of associating with algal lipids, including, but not limited to, Nile Red or Bodipy stain.

Some aspects, may include a kit for analyzing lipid production in algal cultures including, but not limited to, at least one container for a sample; at least one lipid control sample; at least one algal control sample and at least one fluorescent dye (e.g. Nile Red, Bodipy stain). In addition, a kit for detecting lipid production in algae may include a portable fluorescent emission detecting device for facilitating analysis of lipids in the field.

Separation of Algae And Extraction of Oil

In some embodiments, photosynthetic organisms may be separated from the medium and various components, such as oil, may be extracted using any method known in the art. For example, algal organisms may be partially separated from the medium using a standing whirlpool circulation, harvesting vortex and/or sipper tubes or other methods known in the art. Alternatively, industrial scale commercial centrifuges of large volume capacity may be used. Such centrifuges may be obtained from known commercial sources (e.g., Cimbria Sket or IBG Monforts, Germany; Alfa Laval A/S, Denmark). Centrifugation, sedimentation and/or filtering may also be of use to purify oil from other algal components. Separation of algae from the aqueous medium may be facilitated by addition of flocculants, such as clay (e.g., particle size less than 2 microns), aluminum sulfate, FeC13 at pH about pH 9-10, polyacrylamide or the like. In the presence of flocculants, algae may be separated by simple gravitational settling, or flotation, or may be more easily separated by centrifugation. Flocculant-based separation of algae is disclosed, for example, in U.S. Patent Appl. Publ. No. 20020079270, incorporated herein by reference.

A skilled artisan will recognize that any method known in the art for separating cells, such as algae, from liquid medium may be utilized. For example, U.S. Patent Appl. Publ. No. 20040121447 and U.S. Pat. No. 6,524,486, each incorporated herein by reference, disclose a tangential flow filter device and apparatus for partially separating algae from an aqueous medium. Other methods for algal separation from medium have been disclosed in U.S. Pat. Nos. 5,910,254 and 6,524,486, each incorporated herein by reference. Other known methods for algal separation and/or extraction may also be used.

In other embodiments, microorganisms may be disrupted to facilitate separation of oil and other byproduct. Any method known for cell disruption may be utilized, such as ultrasonication, French press, osmotic shock, mechanical shear force, cold press, thermal shock, rotor-stator disruptors, valve-type processors, fixed geometry processors, nitrogen decompression or any other known method. High capacity commercial cell disruptors may be purchased from known sources. (E.g., GEA Niro Inc., Columbia, Md.; Constant Systems Ltd., Daventry, England; Microfluidics, Newton, Mass.) Methods for rupturing microalgae in aqueous suspension are disclosed, for example, in U.S. Pat. No. 6,000,551, incorporated herein by reference. Any other known method for disruption is contemplated herein.

### Conversion of Algae Into Biodiesel

A variety of methods for conversion of photosynthetic derived materials into biodiesel are known in the art and any such known method may be used. For example, algae may be harvested, separated from the liquid medium, lysed and the oil content separated. The algal-produced oil can be rich in triglycerides. Such oils may be converted into biodiesel using well-known methods, such as the Connemann process (see for example, U.S. Pat. No. 5,354,878, incorporated herein by reference). Certain standard transesterification processes involve an alkaline catalyzed transesterification reaction between the triglyceride and an alcohol, typically methanol. The fatty acids of the triglyceride are transferred to methanol, producing alkyl esters (biodiesel) and releasing glycerol. The glycerol can be removed and may be used for other purposes, for example, as a source for mixotrophic growth of algae.

Some embodiments herein may involve the use of the Connemann process (U.S. Pat. No. 5,354,878). Connemann process utilizes continuous flow of the reaction mixture through reactor columns, in which the flow rate is lower than the sinking rate of glycerin. This results in the continuous separation of glycerin from the biodiesel. The reaction mixture may be processed through further reactor columns to complete the transesterification process. Residual methanol, glycerin, free fatty acids and catalyst may be removed by aqueous extraction. The Connemann process is well-established for production of biodiesel from plant sources such as rapeseed oil and as of 2003 was used in Germany for production of about 1 million tons of biodiesel per year (Bockey, “Biodiesel production and marketing in Germany,” www.projectbiobus.com/IOPD_E_RZ.pdf).

However, the skilled artisan will realize that any method known in the art for producing biodiesel from triglyceride containing oils may be utilized, for example as disclosed in U.S. Pat. Nos. 4,695,411; 5,338,471; 5,730,029; 6,538,146; 6,960,672, each incorporated herein by reference. Alternative methods that do not involve transesterification may also be used. For example, by pyrolysis, gasification, or thermochemical liquefaction may be used (see, for example, Dote, 1994, Fuel 73:12; Ginzburg, 1993, Renewable Energy 3:249-52; Benemann and Oswald, 1996, DOE/PC/93204-T5).

In other embodiments, bacterial organisms may be grown in wastewaters or brine waters disclosed herein for the production of biofuels. For example, Pseudomonas species capable of growth using produced water can be harvested for biomass and biofuel produced from the biomass or other products known in the art may be generated using these microorganisms.

### Other Algal Products

The skilled artisan will realize that any type of free-living or symbiotic assembly of algae may be grown, harvested and utilized by embodiments disclosed herein. (See U.S. Pat. Nos. 6,156,561 and 6,986,323, each incorporated herein by reference.). It is contemplated herein that lipids may be separated from algal cultures and other products disclosed herein can be harvested simultaneously, before or after lipid harvesting.

In still further embodiments, other kits are contemplated herein. In some embodiments, a kit may be used for detection of lipid production, extraction or harvesting. Kits can include one or more suitable container means, fluorescent dyes, extraction or harvesting agents. Other kits contemplated of use for embodiments disclosed herein can contain photosynthetic organism inoculums or bacterial culture inoculums. Certain embodiments are directed toward kits having inoculums of microorganisms for growth in wastewaters such as brine waters or brewery waters. Other components contemplated for kits contemplated may include one or more supplements or for example, a predetermined level of one or more nutrients for a culture. Predetermined levels of one or more nutrients may be a mixture of salts, nitrates, phosphates or other nutrient/supplements needed for growing the microorganism.

### EXAMPLES

The following examples are included to demonstrate certain embodiments presented herein. It should be appreciated by those of skill in the art that the techniques disclosed in the Examples which follow represent techniques discovered to function well in the practices disclosed herein, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope herein.

### Example 1

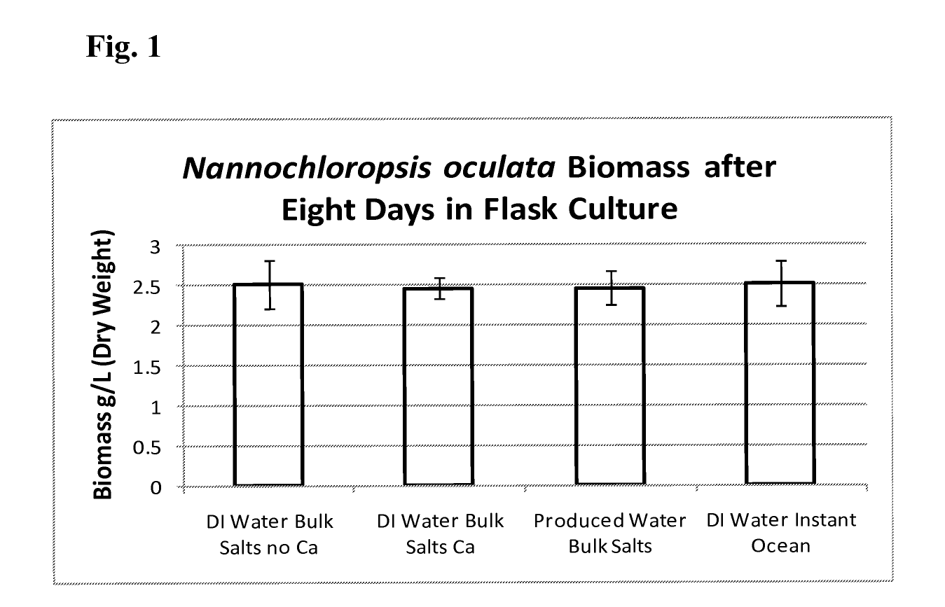

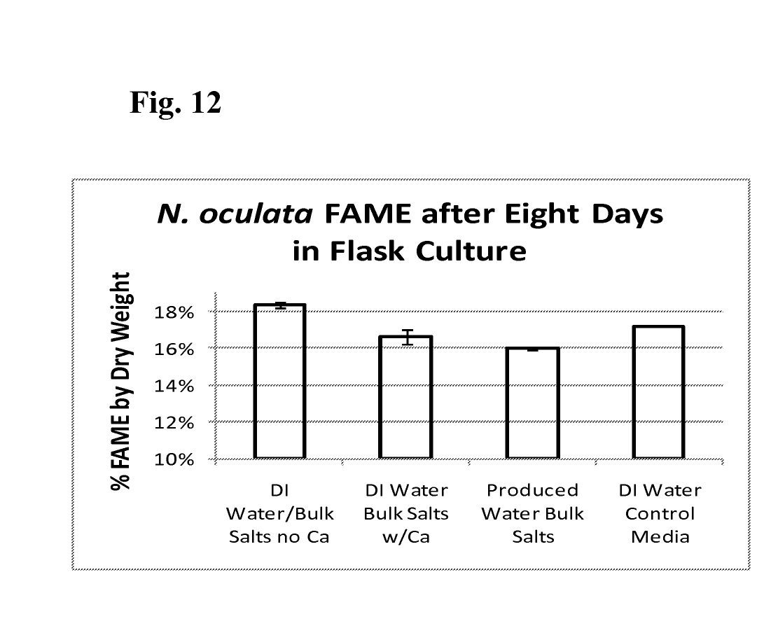

FIG. 1 represents N. oculata (e.g. CCPC number 525, Maine) biomass production after 8 days in culture. Error bars represent the standard deviation of triplicate replicates. One experiment was performed to determine some salt components capable of providing optimal growth conditions in produced water. The media was made with produced water identified in ‘Methods’ and used the bulk salts #1. Treatments with and without calcium were also compared (FIG. 12). These results indicate that N. oculata does not require additional calcium to support growth and may inhibit byproduct or co-product production—therefore this component can be eliminated from the bulk salts recipe. High levels of calcium can induce precipitation of bicarbonate. There are already low concentrations of calcium present in the produced water as compared with seawater so reducing these levels or eliminating them may be another method used depending on the level of calcium. FIG. 1 illustrates that no loss of biomass in the cultures grown in the adjusted produced water compared with those grown in DI water media made with Instant Ocean.

### Example 2

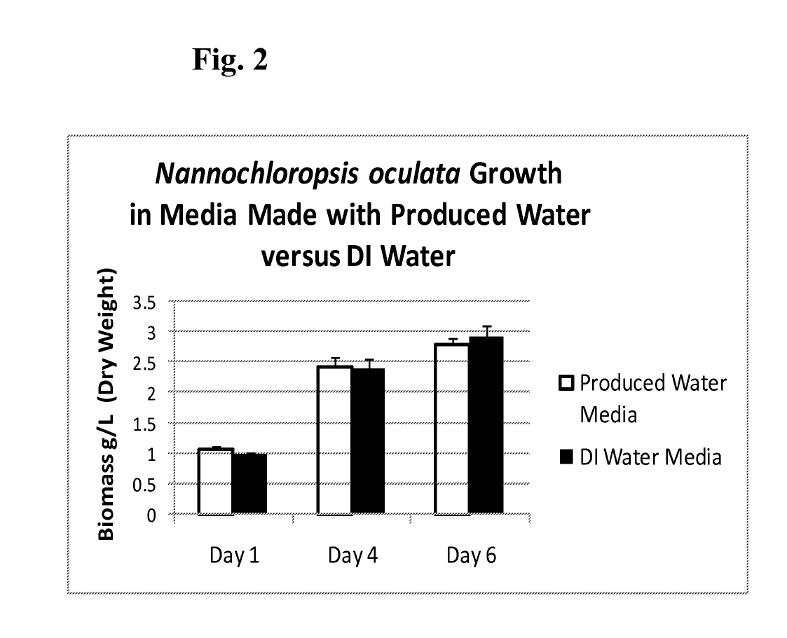

FIG. 2 represents N. oculata growth in media with produced water compared to media with DI water. This experiment used produced water found in ‘Methods’ and bulk salts #1. FIG. 2 illustrates that no significant difference in growth was identified in these experiments. These experiments represent replicates of cultures grown in media made with produced water versus media made with DI water. As illustrated, by Day 4 both cultures had grown to a density of about 2.4 g/L with 0.15 standard deviations between replicates. By Day 6 the mean density of the produced water samples was about 2.8 g/L +/−0.12 and that of the DI water was about 2.9 g/L +/−0.17.

### Example 3

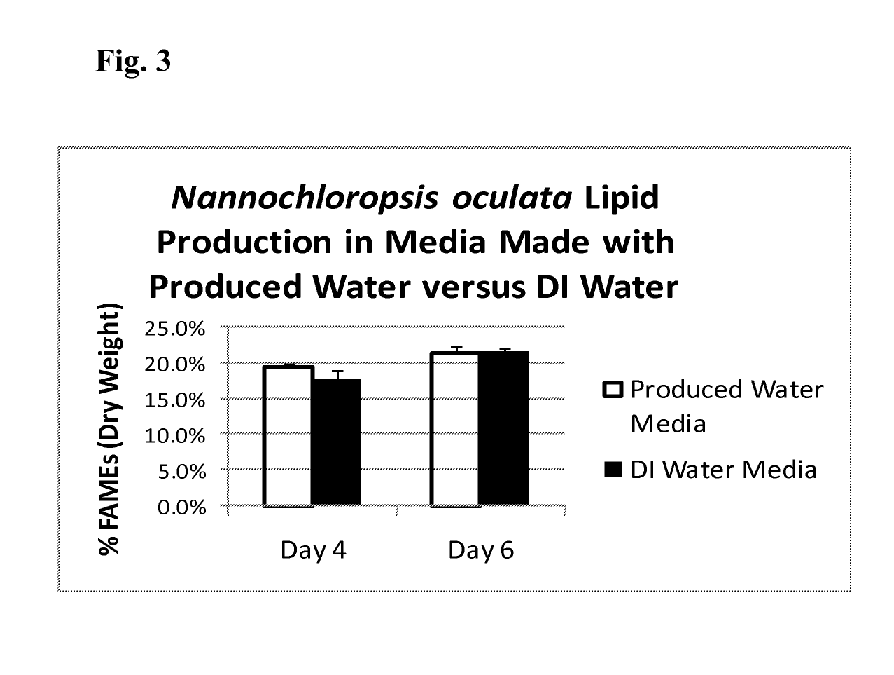

FIG. 3 represents N. oculata lipid production in media made with produce water vs. DI water. This experiment demonstrates that there is no significant difference in lipid production as measured by % FAMEs (Dry Weight) of triplicate reps of cultures grown in media made with produced water versus media made with DI water. Day 4 illustrates a lower % FAME in the DI water media samples versus the produced water (17.7% and 19.5% respectively). This experiment illustrates that lipid production in produced water compared to DI water may not be affected by the produced water and may be slightly increased in certain circumstances and under certain conditions, for example removal or addition of certain supplements (e.g. calcium).

### Example 4

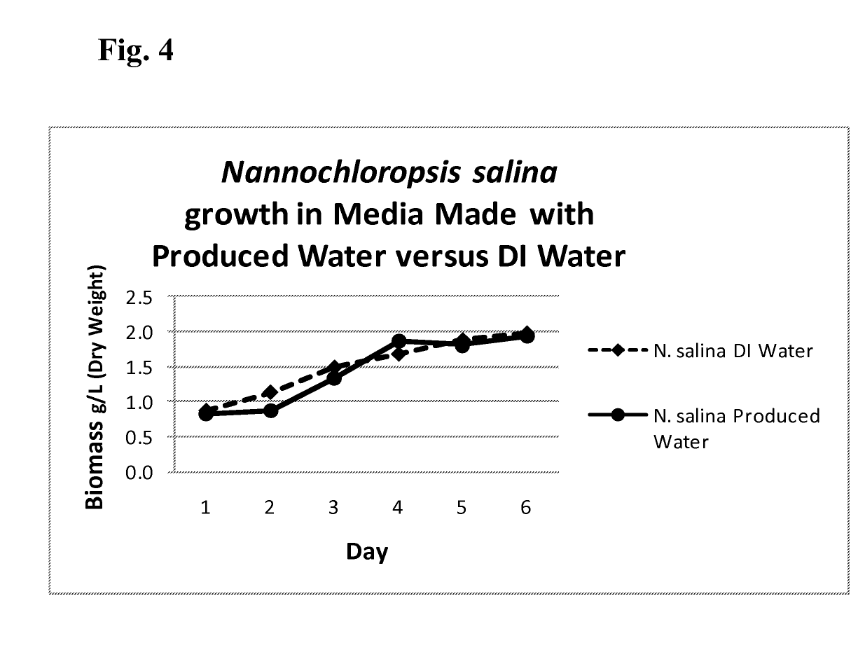

FIG. 4 represents N. salina Growth in Media Made with Produced Water versus DI Water. This figure shows that cultures of N. salina can also be grown in media made with produced water without any significant loss of biomass as compared with the same cultures grown in media made with DI water. These cultures were grown in produced water (a second batch) with bulk salts #2. Any other photosynthetic species or strains capable of use of bicarbonate as an inorganic carbon source are also expected to grow well in media made with produced water.

### Example 5

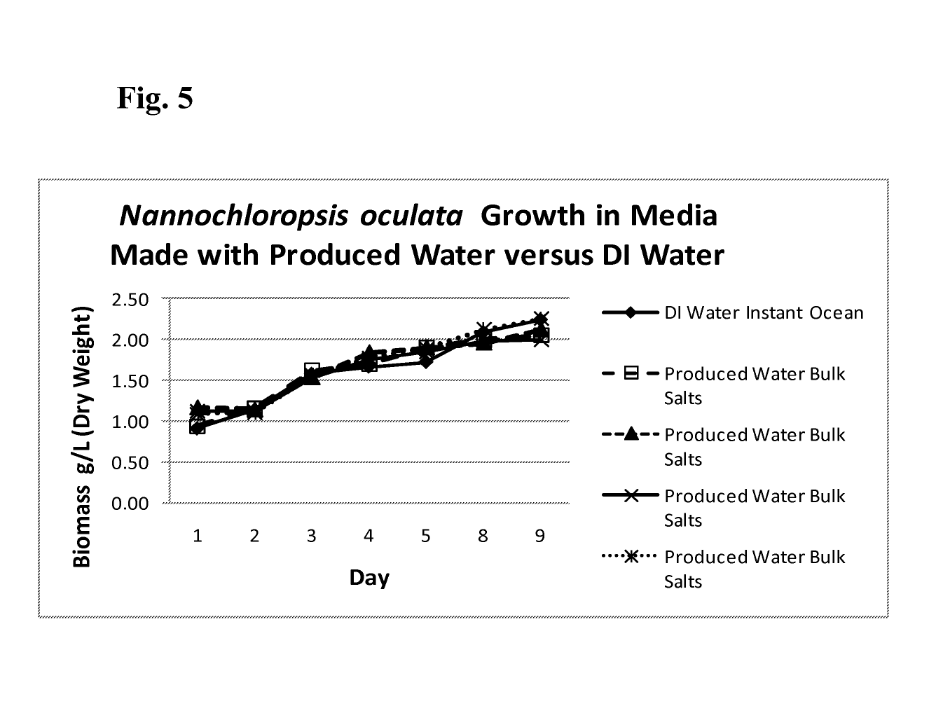

FIG. 5 represents N. oculata growth in media made with produced water versus DI water. These cultures were also grown in produced water, a second sample, with bulk salts #2. This figure illustrates data showing a consistent ability to grow algal cultures in media made with produced water at rates comparable to cultures grown in media made with DI water.

### Example 6

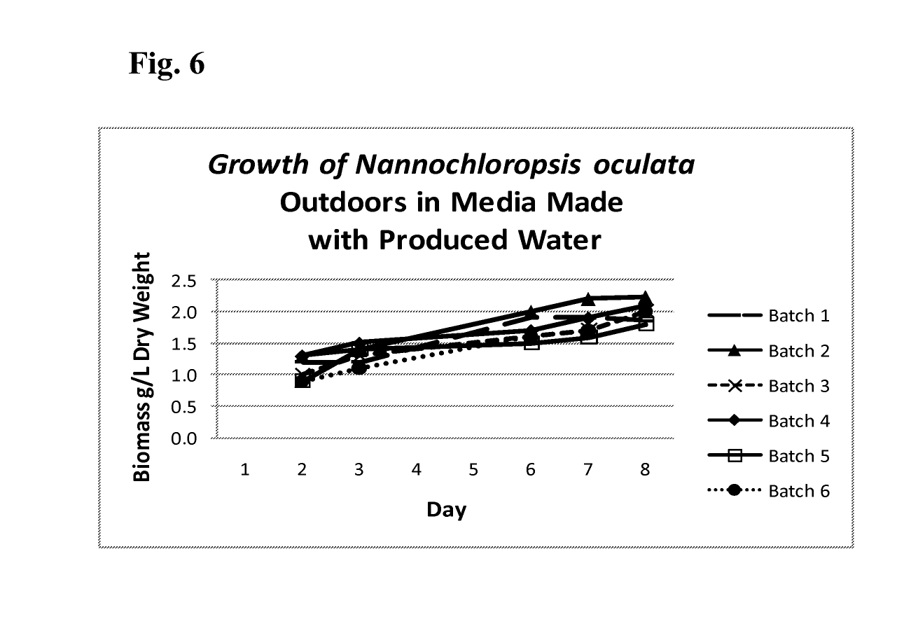

FIG. 6 represents growth of N. oculata outdoors in media made with produced water. This figure illustrates reproducible and predictable growth of N. oculata in produced water under natural sunlight scaled up to about 5 liter cultures. These batches were grown in media made with about 5 liters of produced water bulk salts #1.

### Example 7

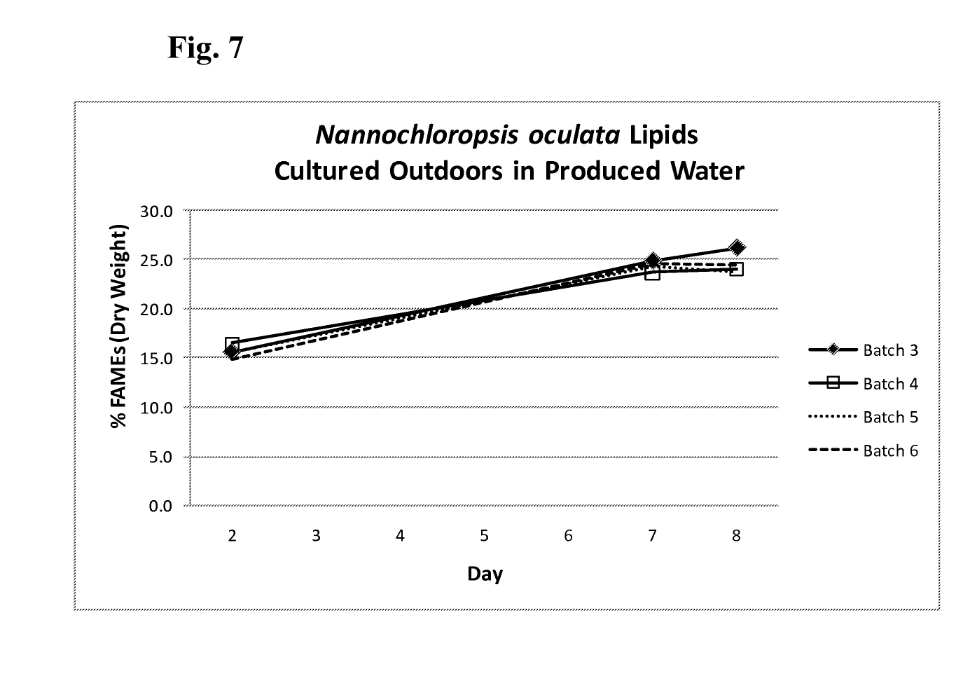

FIG. 7 illustrates Nannochloropsis oculata lipids harvested from growth in produced water. FIG. 7 illustrates an increase in lipids generated by N. oculata as determined by % FAMEs on a dry weight basis during the course of eight days of incubation in media made with produced water. The growth curves associated with this are presented in FIG. 6.

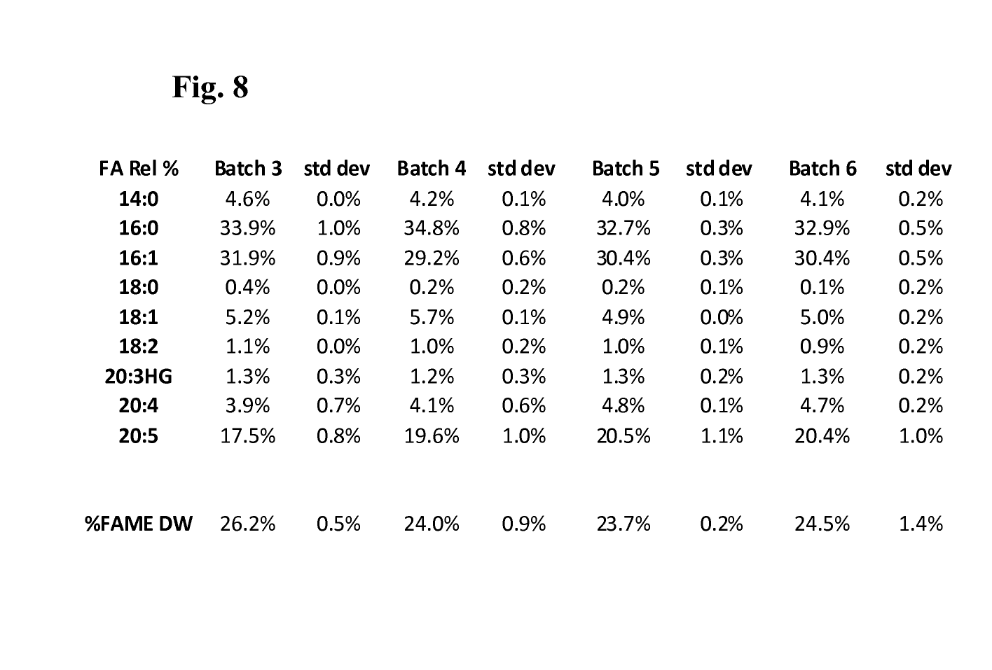

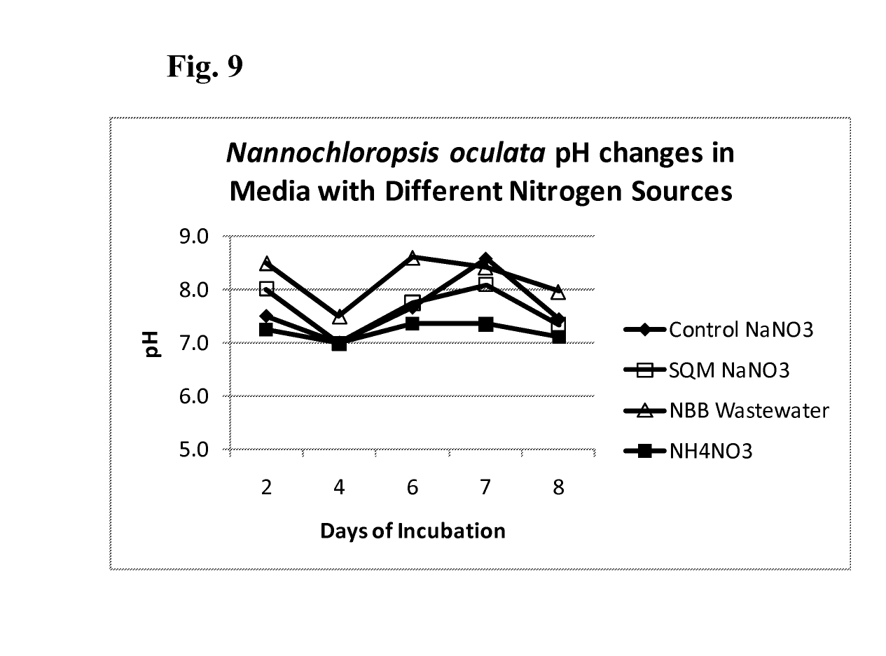

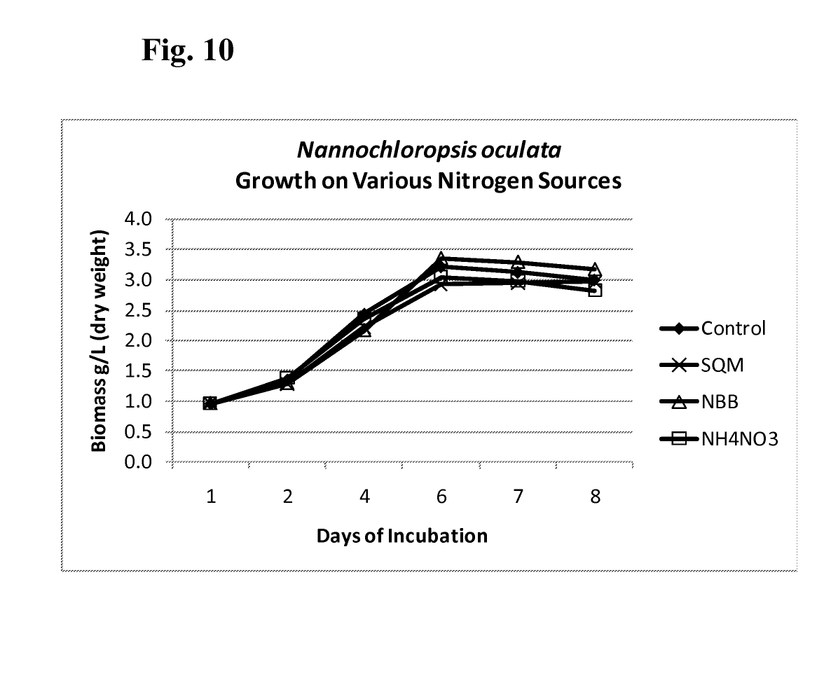

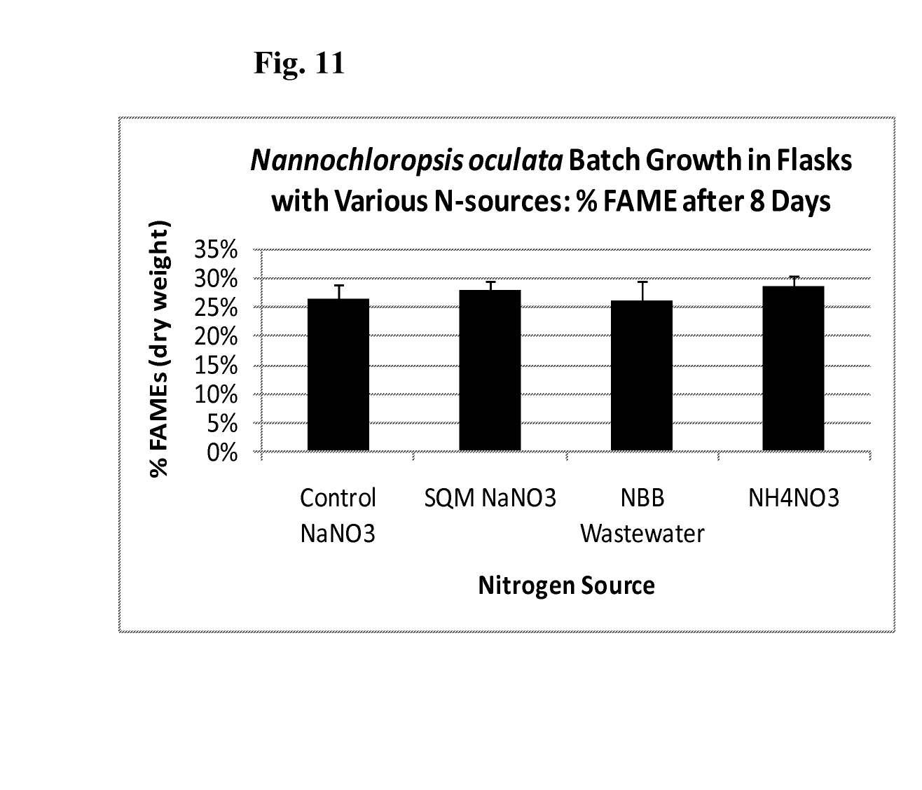

FIG. 8 represents a chart of the fatty acid profiles from the cultures described in FIG. 6 and FIG. 7. This chart represents relative percent of each type of fatty acid of the total % FAMES by Dry Weight (DW) after eight days growth in media made with produced water. The mean total % FAME by dry weight for the four batches is 24.6+/−0.01 which demonstrates reproducibility and consistency. The distribution of the fatty acids is consistent with batches grown in the current production media under similar conditions. FIG. 9 illustrates pH change over an 8 day period with various nitrogen supplements indicated. It was observed that the pH of brewery wastewater (NBB) was typically higher when compared to a control and 2 other test compositions over the 8 day period yet the increase in pH did not seem to affect growth of the microorganisms (FIG. 10) and lipid production (FIG. 11).

### Methods

Some exemplary methods have supplements added to medias for growth of microorganisms. In certain methods, nutrients added to a culture of microorganisms or media used for culturing can be 425 mg/L sodium nitrate fertilizer; 50 mg/L monopotassium phosphate fertilizer; trace metals at 1 ml/L per the f/2 recipe of Guillard, 1975. Some of the cultures were inoculated to an initial density of 1 g/L where the increase in introduced density reduced lag time. Other methods used predetermined high density inoculums that permit reduced growth time without the need for additional nutrients. For example, some of the algal cultures were started using an inoculation in media of about 1.5 g/L (wet pellet/media) which was shown to increase yield without the need for additional nutrients (data not shown). Salts used as media supplements may be from, for example, produced water used alone or as a supplement to other water or media composition, commercial sources, or Instant Ocean. Salt was added to some media to attain 16 ppt salinity. Many of the photosynthetic organisms can be cultured in a range of salinities, for example, Nannochloropsis can grow in about 5 ppt to about 40 ppt. Media made with DI or tap water has a pH 7.2-7.3 whereas media made from the produced water can range from pH 8.0-8.3. Microorganisms, such as photosynthetic organisms, can grow well at a wide range of pH ranges. Nannochloropsis can be grown in about pH 7 to about pH 9 (see for example FIG. 9).

### Bulk Salts #1

In some embodiments, certain salts were used to supplement cultures or medias for growing cultures. In one media, salts used included sodium chloride 13.8 g/L (e.g. Cargill Hi-grade evaporated salt); magnesium chloride 3.5 g/L (e.g. North American Salt Company Freezeguard Crystals); and potassium magnesium sulfate 2.0 g/L (e.g. K-mag fertilizer 0-0-22); were used. Optionally: 0.6 g/L calcium chloride; 0.1 g/L sodium bicarbonate (e.g. Solvay Chemicals) may be used to bring salinity to about 16 ppt but the same or similar ratio can be used to increase or decrease total salinity as desired. Potassium magnesium sulfate was not completely soluble and tended to quickly clog filters used to sterilize the media. Therefore, another media mixture was formulated to replace this ingredient with a combination of magnesium sulfate and potassium chloride. No difference in growth was associated with the switch to the new recipe therefore they could be used interchangeably. In other methods, bicarbonate does not need to be added to produced water and calcium may not be necessary for growth and in some cases inhibited production and/or harvesting of lipids compared to media without calcium.

### Bulk Salts #2

In other experiments, a different media mixture for growing algal cultures was used. This media mixture contained salts of sodium chloride 13.3 g/L (e.g. Cargill Hi-Grade Evaporated Salt); 2.42 g/L MgCl₂.6H₂O (e.g. North American Salt Company Freezeguard Crystals); magnesium sulfate 3.36 g/L (e.g. Epsom Salts), and potassium chloride 0.37 g/L (e.g. Compass Minerals water softener pellets).

Modified f/2 media: 69.7 mg/L Nitrogen-N from sodium nitrate or other nitrogen source; 34.5 mg/L PO₄⁻from monopotassium phosphate or other soluble phosphorous source; (no calcium compound), trace metals as per f/2 media: 180 μg/L MnCl₂, 22 μg/L ZnSO₄.7H₂O, 9.8 μg/L CuSO₄.5H₂O, 10 μg/L CoCl₂.6H₂O, 6.3 μg/L NaMoO₄, 4.36 mg/L Na₂EDTA, 3.15 mg/L FeCl₃.6H₂O.

The modified f/2 media has been optimized for 1-1.5 g/L initial culture density with 600 uE of light or greater at temperatures between 20-25 C and will yield 3-5 g/L biomass within one to two weeks respectively. The recipe is modified further for specific environmental conditions with less nutrients required under lower light or temperature and more nutrients required for more dense cultures or higher light or temperature. It is recommended to begin with 1 g/L initial culture density or greater which overcomes or significantly reduces lag stage and inhibits growth of biological contaminants. Addition of 1× N&P doses at 1.5 g/L, 3.0 g/L and 4.5 g/L culture density can induce an increase to 7 g/L within about two weeks partially due to extension of log phase during batch culture but will also affect product, co-product and byproduct composition. Inorganic carbon can be supplied from carbon dioxide or bicarbonate and pH typically should be maintained above 7 and below 8.5 for optimum growth. The amount of inorganic carbon of use is often relative to the available light and nutrients.

### Produced Water

Produced water used in certain exemplary experiments contained high concentrations of sodium bicarbonate. Alkalinity during the course of the experiments ranged from 2200 ppm to over 4000 ppm. In some experiments, Nannochloropsis was grown in produced water. Nannochloropsis is capable of using bicarbonate as an inorganic carbon source. Using produced water high in bicarbonate can reduce or eliminate the need to sparge with carbon dioxide as an inorganic carbon source. Supplying carbon dioxide is one of the more costly requirements for growth of algae by other methods. Produced water samples were tested for volatile organics which were present in concentrations <1 part per million (ppm). Produced water in these experiments were supplemented with commercial salts as needed to a salinity of 16 parts per thousand (ppt). Diesel range organics were present in concentrations up to 10 ppm were, in some experiments, removed by filtration. Some of the algal microorganisms can grow in the presence of produced water contaminants. For example, Nannochloropsis has previously been reported to be tolerant of diesel fuel.

FIGS. 1-3 represent data generated from experiments conducted in 250 ml glass flasks incubated indoors under approximately 200 uE light for 16 hours/day at 21-25C. Flasks were sparged with approximately 1-2% carbon dioxide mixed in air and shaken continuously. Triplicates of each treatment were inoculated to an initial density of approximately 1 g/L from an algal pellet into a total volume of 100 mls media. Biomass was monitored by optical density at 750 nm on a HACH spectrophotometer and the data was transformed to g/L using a previously determined coefficient. Cultures were monitored by microscopy and flow cytometry for contamination. Biomass data was corrected for evaporation by measurement of volume and conductivity. FAME data was obtained by in-situ transesterification of a previously frozen and thawed, wet pellet by mild methanolysis and heptane extraction followed by Gas Chromotography on an Agilent 7890A with a FAME Wax column.

FIGS. 4-5 represent data generated from experiments conducted in 2 L Fernbach flasks incubated indoors under approximately 200 uE light for 16 hours/day at 21-25 C. Flasks were sparged with approximately 1-2% carbon dioxide mixed in air and shaken continuously. Each treatment was inoculated to an initial density of approximately 1 g/L from an algal pellet into a total volume of 1000 mls media. Biomass was monitored by Flow Cytometry on a Guava Easy Cyte Plus and the data was transformed to g/L using a previously determined coefficient. Evaporation was controlled by daily addition of DI water to the flasks to return to initial volume.

### Methods For Outdoor Growth In An Algal Growth System (AGS)

FIGS. 6-8 represent data from outdoor cultures in about 5 liter AGS plastic panels in a solar basin at a temperature of 21-24° C. They were inoculated to an initial density of approximately 1 g/L from culture previously acclimated to produced water. The pH was maintained between about 7.5 to about 8.3 with sparging from approximately 5% carbon dioxide and air. Cultures were grown in northern Colorado in March under natural sunlight. Cultures were monitored by optical density, flow cytometry and microscopy and FAME content determined by the method described above. Biomass density was corrected for evaporation by use of conductivity data.

Table 1 represents an example of potential reductions in expenses when using fertilizer versus lab grade nutrients and substitution of bulk salts mix for Instant Ocean and other substitutions for supplements. The recipe is for substitution of half-strength seawater (16 ppt) which was used in most of the experiments represented. For full strength seawater (35 ppt) double the amount (and cost) of the bulk salts or multiply by a fraction to derive water compositions of any desired salinity. Nannochloropsis sp. can be grown in a wide range of salinity from 5 ppt to over 40 ppt. Salinity can be used to manipulate products, co-products, byproducts and to control the types of biological contaminants such as bacteria, other algae and protozoa. A substitution of KMag for MgSO4 (potassium magnesium sulfate) and KCl was used for some experiments. Some experiments also added calcium chloride and/or sodium bicarbonate to the bulk salts depending on an inorganic carbon source supplied. Calcium chloride was demonstrated to reduce the yield of FAMEs and did not increase growth when present in the media, therefore it was removed. Therefore, certain compositions media that has reduced to no calcium, or has a calcium precipitant. Low concentrations of calcium may be accomplished by using tap water, brine water, brewery water or other wastewater. Inorganic carbon can be supplied as carbon dioxide or bicarbonate but Nannochloropsis can use inorganic carbon in the form of bicarbonate. If needed, carbon dioxide will be supplied. In these compositions, these microorganisms are grown at a pH greater than 7 which naturally produces bicarbonate from added carbon dioxide. Any source of nitrogen and phosphorous can be used in these culture medias. The ratio of the nutrients required for optimizing growth, product, byproduct, co-product production depends upon culture density and available light as well as the inorganic carbon source and concentration and the media pH. Nitrogen and phosphorous from wastewater streams can be used at a lower cost than fertilizers. Trace metals can also be found in wastewater streams as well as natural chelators that can further reduce the nutrient costs associated with growing microorganisms. Use of brine water may also partially offset the cost of nitrogen because this nutrient is present in the brine waste along with salts and inorganic carbon.

TABLE 1

| | Nutrient | Media | Cost |
|---|---|---|---|
| | cost/g | g/L | $/L Media |
| Sodium Nitrate | | | |
| Lab Grade | 0.00747 | 0.425 | 0.00317 |
| Fertilizer Grade | 0.00088 | 0.425 | 0.00037 |
| Monopotassium Phosphate | | | |
| Lab Grade | 0.01090 | 0.050 | 0.00055 |
| Fertilizer Grade | 0.00568 | 0.050 | 0.00028 |
| Instant Ocean | 0.00050 | 20.000 | 0.00997 |
| NaCl | 0.00004 | 13.300 | 0.00050 |
| MgCl₂ | 0.00012 | 2.420 | 0.00029 |
| MgSO₄ | 0.00038 | 3.360 | 0.00127 |
| KCl | 0.00012 | 0.370 | 0.00005 |
| Bulk Salts Total | | | 0.00210 |
| Trace Metals | $/mg | mg/L | $/L |
| MnCl₂ | 0.000041 | 0.180 | 0.0000074 |
| ZnSO₄•7H₂O | 0.000021 | 0.022 | 0.0000005 |
| CuSO₄•5H₂O | 0.000047 | 0.010 | 0.0000005 |
| CoCl₂•6H₂O | 0.000062 | 0.010 | 0.0000006 |
| NaMoO₄ | 0.000104 | 0.006 | 0.0000007 |
| FeCl₃•6H₂O | 0.000128 | 3.150 | 0.0004034 |
| Na EDTA | 0.000053 | 4.360 | 0.0002328 |
| | | | 0.0006458 |

### Example 8

In some embodiments, Chilean nitrate (SQM North America Co.) can be used. In other exemplary methods, other fertilizer can be used as a source of nitrogen and/or phosphorus. In other methods it is contemplated that wastes from, for example, a brewery, such as brewery wastewater can be used. Brewery water may be adjusted for the appropriate level of salinity. For example, the salinity may be adjusted by using Instant ocean, brine waste or other salts. In yet other methods, trace metals were used as sources to supplement media for growth of Nannochloropsis oculata and other species. The growth curve for algae in wastewater was identical to the artificially made media supplemented with KNO₃ or NaNO₃ or Chilean nitrate (SQM) or other available cheap fertilizers. In one exemplary method, FAMEs reached 37%-40%.

In other exemplary methods, Nannochloropsis oculata were grown at a very low salinity media, and biomass remained the same as normal salt levels producing FAMEs. Low salinity was about 5 g/L (data not shown).

In another exemplary method, wastewater from breweries can serve several purposes with respect to growing or supplementing microorganisms. Wastewater can be used to grow algae, for generating biofuels from microorganisms and other co-products as well as, use discarded and problematic wastewaters in an efficient manner (see for example, FIGS. 10 and 11). FIG. 10 represents growth of Nannochloropsis oculata in different media; Dry Weight (g/L). FAMES produced by growth in the different medias is represented in FIG. 11.

In one exemplary method, different strains of Nannochloropsis can be grown in an algal growth system to generate lipids. In one method, these species were grown to high cell density at pH 7.0-7.5 controlled by CO₂. This process can be done in one or more photobioreactor production systems (mono- or biphasic approach: PBR-ponds). The amount of lipids produced may vary from about 20 to about 50% FAMEs depending on the conditions.

In another exemplary method, algal cultures were grown in brine water or in other media with brine water as a supplement. There are several advantages in growing algae cultures in brine water. For example, brine water can provide bicarbonate, salts and nutrients. In addition, brine water cost very little to nothing, reducing production costs. Brine wastewater can be included, but is not limited to, a source of sodium, chloride, sulfur, magnesium, calcium, potassium, bicarbonate and nitrogen. One advantage for using brine water is that the cost for ocean salts and carbon dioxide use may be reduced through the use of brine wastewater, for example, as replacement for other salts and inorganic carbon. Brine water may vary from batch to batch but can supply a small amount of salt required for algal cultures or all the salt required for algal growth. In addition, a mix of brine water with other wastewaters may be used. In this case, brine water can be a source of salts or inorganic carbon and wastewater as nutrients (data not shown).

Certain parameters from experiments discussed above may be specific to Nannochloropsis oculata or salina but can also apply to other marine algae capable of growth in modified f/2 media, see for example, some of these listed below and other species disclosed herein.

| Species | Temp (c.) | Size (um) |
|---|---|---|
| Phaeodactylum tricornutum | 11-25 | 12-14 × 2-4 |
| Skeletonema costatum (Greville) Cleve | 11-30 | 6-14 × 6-8 |
| Cyclotella cryptica Reimann, Lewin et. | 11-21 | 10-16 × 12-16 |
| Guillard | | |
| Chaetoceros calcitrans Paulsen | 17-26 | 3-7 × 3-5 |
| Thalassiosira pseudonona (Hustedt) Hasle et | 4-25 | 4-6 × 4-6 |
| Heimdal | | |
| Nannochloropsis salina Hibberd | 17-22 | 2-4 × 2-3 |
| Nannochloropsis salina Hibberd | 11-16 | 2-4 × 2-4 |
| Nannochloropsis gaditana Lubian | 13-22 | 2-5 × 2-8 |
| Nannochloropsis gaditana Lubian | 11-21 | 2-3 × 2-3 |
| Isochrysis galbana | 3-28 | 4-6 × 4-6 |
| Emiliania huxleyi (Lohm) Hay et. Mohler | 8-25 | 3-6 × 3-6 |

### Lipid Detection

In other methods, fluorometric detection assays were developed for lipid analysis in algal cultures such as Nannochloropsis oculata. In one embodiment, fluorescent dye, Nile Red, can be used. One issue to contend with is to get rid of false positives related to lipid detection in previous methods. In order to reduce false positives, algal cells were resuspended in a growth medium and centrifuged for 5 min at 2000 RCF, the supernatant was decanted and pelleted cells were resuspended in fresh media and and diluted to OD 0.1 using Distilled Water. In this example, Nile Red was added to a final concentration of 1 ug/ml in DMSO solution and the composition was vortexed for around 5 minutes. In certain procedures, the cells were osmotically shocked and/or heat shocked to disrupt the cell wall. Then, the Nile Red solution was incubated with algae cells for about 1 h at about 30° C. The signal was measured at excitation 525 nm and emission 580 nm, (data not shown). In certain embodiments, neutral lipids emit at 540-600 nm and polar lipids and chlorophyll emit at wavelength above 600 nm. Other similar experiments have been performed using Bodipy stain for lipid detection in algal organisms (data not shown).

### Methods For Harvesting Lipids From Algal Cultures

In certain exemplary methods, improved methods of transesterification were developed. In this example, the method was able to accommodate wet or dry pellets of algae. Little difference was observed between extracting oil from wet pellets or dry ones. This is advantageous for scale-up and production because no excess energy is needed for dry algae. In this example, 2.5 mL of 0.2 N KOH in methanol was added to a 5 mg algae wet pellet, mixed and transferred into the glass test tube. Another 2.5 mL of 0.2 N KOH in methanol was dispensed into the centrifuge tube, mixed and transferred to the glass test tube and cap glass tube with Teflon lined cap. 5 mL of 0.2 N KOH in methanol was used as a blank. 5 mL of 0.2 N KOH in Methanol with 2 μl of Flax oil served as a control. All samples were vortexed for about 20 seconds, and incubated for about one hour at 37° C. with an additional vortexing samples for 10 sec. every 10 minutes. After removal of tubes from heat 1 mL 1 M acetic acid and 2 mL HPLC grade hexane was added and samples were vortexed for about 20 seconds. Samples are centrifuged for about 5 minutes at 2000 RCF and 25° C. and top hexane layer or the organic layer was removed and transferred to GC vial for analysis. GC results for Nannochloropsis oculata profile are not shown.

### Example 9

### Base Catalyzed Transesterification Protocol For Creating Fatty Acid Methyl Esters (FAME)

### Small Scale Reaction For High Thoughput Sample Analysis

Sample collection: A dry mass (g/L) of the culture is estimated via for example, spectrophotometry at OD₇₅₀ nm, based on a correlation coefficient from empirical data of actual dry weight versus OD₇₅₀ nm. An equivalent volume of algae is centrifuged to obtain a 5 mg pellet, the supernatant removed, and the pellet frozen at −20° C., under nitrogen until the reaction is performed.

In this procedure, the transesterification was optimized for sample size to reagent stoichiometry for FAME yield, reduced time for process and use of a wet versus dry pellet.

Reaction: The sample is thawed to approximately room temperature, 5 ml of 0.2M Potassium Hydroxide in Methanol is added to the sample (2.5 mL twice) and transferred to an acid washed glass tube with Teflon lined cap. The sample is vortexed and heated to 37° C. for 1 hour, vortexing every 10 minutes. The protocol has been further optimized to reduce reaction time to 30 minutes or less for high throughput. The sample is removed from heating and 1 mL of 1M acetic acid is added to stop the transesterification reaction, 2 mL of Heptane are added to extract the FAMEs, and the sample is vortexed. It was found that a wet pellet of algae could be used in certain ratios of about 5 mgs to 5 mls of Potassium Hydroxide in Methanol and successful transesterification could be performed saving about 50% of the time from previous preparations. In addition, use of heptane versus hexane permitted a more efficient sample preparation and lipid analysis process.

Separation: The sample is centrifuged to affect a separation of the aqueous and organic phases. The organic phase is removed for analysis. A 1:10 dilution is done on the organic fraction. A 23:0 FAME internal standard is added for quantification and the samples are then analyzed with a gas chromatograph (GC) with appropriate external standards.

### Methods For Flask Experiments

In one experiment, an investigation was conducted regarding feasibility of using commercial fertilizers and wastewater streams from breweries to reduce the cost of nutrients and salts used to make growth media for growing marine algae for production of lipids and other products. Brewery wastewater can be an alternative source of nitrogen, phosphorus and trace metals. Brewery wastewater was obtained from a couple of microbreweries post wastewater treatment at their facility. The water was filtered and supplemented with 20 g/L Instant Ocean Salts and 1 ml/L Gullard Trace Metal Mix (data not shown).

Experiments were conducted using brewery wastewater from microbreweries as the base water for media with minimal supplements added to grow algae. In these experiments, brewery waste water was obtained, supplemented with 20 g/L Instant Ocean salts and 1 ml/L Guillard Trace Metal Mix. The water was then filter sterilized and inoculated with Nannochloropsis oculata and grown for a period of time to evaluate growth and lipid production.

In one experiment, 500 ml glass Erlenmeyer flasks were incubated indoors under approximately 200 μE/m²/s light on a 16:8 light:dark schedule at 21-25° C. Cultures in flasks were sparged directly with approximately 1-2% carbon dioxide mixed in air and shaken continuously. Each treatment was performed in triplicate. The three treatments in this experiment consisted of: a) control treatment of Nannochloropsis oculata grown in f/2 growth media, b) brewery wastewater following wastewater treatment from microbrewery 1, and c) wastewater from the beer brewing process at microbrewery 2. Each of the wastewaters were filtered and 20 g/L Instant Ocean salts and 1 ml/L Guillard trace metals mix were added. Each triplicate treatment was inoculated with approximately 1 g/L Nannochloropsis oculata and grown for seven days. Algae growth was evaluated by measuring optical density at 750 nm on a Hach spectrophotometer and data was converted to g/L using a previously determined coefficient. Evaporation was corrected for by adding sterile DI H₂O back to the original volume periodically and before measurements were taken (data not shown).

FIG. 12 illustrates an increase in FAMEs from samples cultured without calcium (Ca). In some methods, if for example, lipid production is sought, a media composition without any or with a reduced level of calcium is recommended, based on an increase in lipid harvested from cultures without calcium. Other culture supplements had calcium supplied with the salts or from the produced water. Some water compositions (e.g. brine waters) naturally contain calcium and may be put through a process or treated with a compound capable of precipitating or removing the calcium at the same time, after, or prior to, combining the water composition with a culture in order to increase product or byproduct production in a culture of microorganisms.

All of the COMPOSITIONS and/or METHODS and/or APPARATUS disclosed and claimed herein can be made and executed without undue experimentation in light of the present disclosure. While the compositions and methods of this invention have been described in terms of preferred embodiments, it will be apparent to those of skill in the art that variation may be applied to the COMPOSITIONS and/or METHODS and/or APPARATUS and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition comprising, an introduced culture of microorganisms, a brine water composition and at least one supplement.

2. The composition of claim 1, wherein the introduced culture of microorganisms comprises a culture of photosynthetic organisms or a culture of bacteria.

3. The composition of claim 2, wherein the introduced culture of photosynthetic organisms comprises one or more cultures of algal strains.

4. The composition of claim 1, wherein the brine water composition comprises produced water.

5. A composition comprising, a brewery wastewater composition, an introduced culture of photosynthetic microorganisms and at least one supplement.

6. The composition of claim 5, wherein the introduced culture of photosynthetic organisms comprises one or more cultures of algal strains.

7. The composition of claim 5, further comprising a brine water composition.

8. The composition of claim 7, wherein the brine water composition and the brewery wastewater are a mixture of a predetermined ratio capable of growing the photosynthetic microorganisms.

9. A method for growing introduced photosynthetic organisms in culture comprising:
obtaining a water composition selected from a brine water composition, a brewery wastewater or a mixture thereof and growing the introduced photosynthetic organisms in the water composition.

10. The method of claim 9, further comprising adding one or more supplements to the culture.

11. The method of claim 9, wherein the introduced photosynthetic organisms comprises one or more cultures of algal strains.

12. The method of claim 9, further comprising harvesting one or more product, byproduct or co-product from the cultures.

13. A system comprising: providing a brine water composition to a culture of microorganisms wherein the system delivers the brine water to the culture.

14. The system of claim 13, further comprising a sieve component for removing particulates from the brine water prior to the brine water contacting the culture.

15. The system of claim 13, further comprising a component for adding one or more supplements to the culture of microorganisms.

16. The system of claim 13, further comprising a culture vessel for culturing the microorganisms.

17. The system of claim 13, further comprising a means for harvesting the microorganisms.

18. A method for reducing length of lag-phase for growing photosynthetic microorganisms comprising:
introducing an inoculum of a photosynthetic organism to a media wherein the inoculum and the media comprises a density of about 1 gram wet pellet of photosynthetic organism per liter of media to about 5 grams wet pellet of photosynthetic organism per liter of media; and
harvesting the algal culture in either log or stationary phase of the culture after introduction of the inoculum.

19. The method of claim 18, further comprising harvesting one or more products, byproducts or co-products from the cultures.

20. The method of claim 18, wherein the inoculum comprises a photosynthetic microorganism comprising Nannochloropsis oculata, Nannochloropsis sp., Nannochloropsis salina, Nannochloropsis gaditana, Tetraselmis suecica, Tetraselmis chuii, Chlorella sp., Chlorella salina, Chlorella protothecoides, Chlorella ellipsoidea, Chlorella emersonii, Chlorella minutissima, Chlorella pyrenoidosa, Chlorella sorokiniana, Chlorella vulgaris, Chroomonas slaina, Cyclotella cryptic, Cyclotella sp., Dunaliella tertiolecta, Dunaliella salina, Dunaliella bardawil, Botryococcus braunii, Euglena gracilis, Gymnodimium nelsoni, Haematococcus pluvialis, Isochrysis galbana, Monoraphidium minutium, Monoraphidium sp., Nannochloris, Neochloris oleoabundans, Nitzschia laevis, Onoraphidium sp., Pavlova lutheri, Phaeodactylum tricornutum, Porphyridium cruentum, Scenedesmus obliquus, Scenedesmus quadricaula, Scenedesmus sp., Stichococcus bacillaris, Stichococcus minor, Spirulina platensis, Thalassiosira sp., Chlamydomonas reinhardtii, Chlamydomonas sp., Chlamydomonas acidophila, Isochrysis sp., Phaeocystis, Aureococcus, Prochlorococcus, Synechococcus, Synechococcus elongatus, Synechococcus sp., Anacystis nidulans, Anacystis sp., Picochlorum oklahomensis, Picocystis sp. or a combination thereof.

21. A kit for generating rapid production of algae biomass comprising;
a wet pellet of one or more algae microorganisms for making initial culture density of 1 gram wet pellet of photosynthetic organism per liter of media to about 5 gram wet pellet of photosynthetic organism per liter of media;
lyophilized modified f/2 media composition;
one or more containers; and
optionally, one or more predetermined supplements for growth in brine water or brewery wastewater.

22. The kit of claim 21, wherein the modified f/2 media comprises a media having no calcium and optionally, a calcium precipitant.

23. The kit of claim 21, wherein the algal cultures comprise Nannochloropsis oculata, Nannochloropsis sp., Nannochloropsis salina, Nannochloropsis gaditana, Tetraselmis suecica, Tetraselmis chuii, Chlorella sp., Chlorella salina, Chlorella protothecoides, Chlorella ellipsoidea, Chlorella emersonii, Chlorella minutissima, Chlorella pyrenoidosa, Chlorella sorokiniana, Chlorella vulgaris, Chroomonas slaina, Cyclotella cryptic, Cyclotella sp., Dunaliella tertiolecta, Dunaliella salina, Dunaliella bardawil, Botryococcus braunii, Euglena gracilis, Gymnodimium nelsoni, Haematococcus pluvialis, Isochrysis galbana, Monoraphidium minutium, Monoraphidium sp., Nannochloris, Neochloris oleoabundans, Nitzschia laevis, Onoraphidium sp., Pavlova lutheri, Phaeodactylum tricornutum, Porphyridium cruentum, Scenedesmus obliquus, Scenedesmus quadricaula, Scenedesmus sp., Stichococcus bacillaris, Stichococcus minor, Spirulina platensis, Thalassiosira sp., Chlamydomonas reinhardtii, Chlamydomonas sp., Chlamydomonas acidophila, Isochrysis sp., Phaeocystis, Aureococcus, Prochlorococcus, Synechococcus, Synechococcus elongatus, Synechococcus sp., Anacystis nidulans, Anacystis sp., Picochlorum oklahomensis, Picocystis sp. or a combination thereof.

24. The kit of claim 21, wherein the wet pellet is at least partially dehydrated.

25. The kit of claim 21, wherein the algal culture comprises a selected mutant algal culture capable of growing in a brine water.

26. The kit of claim 21, wherein the algal culture comprises a selected mutant algal culture capable of growing in a brewery wastewater.

27. A composition for affecting lipid production in algal cultures comprising; a modified f/2 media having no calcium compound and optionally, a calcium precipitant, the modified f/2 media affects lipid production compared to control media.

28. A method for affecting lipid production in algal cultures comprising: growing an algal culture in a modified f/2 media having no calcium; and harvesting lipids from the algal cultures, the modified f/2 media affects lipid production compared to control media.
